(19) United States
(12) Patent Application Publication
(10) Pub. No.: US 2025/0288721 A1
(43) Pub. Date: Sep. 18, 2025

(54) TISSUE STRUCTURE ISOLATION USING A MICRO-CUTTING DEVICE AND USES OF RESULTING MICRO-CUBES OF TISSUE

(71) Applicant: Jörg C. GERLACH, Berlin (DE)

(72) Inventor: Reinhard BORNEMANN, Bielefeld (DE)

(21) Appl. No.: 18/860,365

(22) PCT Filed: Apr. 26, 2023
(86) PCT No.: PCT/EP2023/061020

§ 371 Date: Oct. 25, 2024

(30) Foreign Application Priority Data
Apr. 27, 2022 (WO) PCT/EP2022/061179

(51) Int. Cl. A61L 27/36; A61L 27/54

(52) U.S. Cl. CPC A61L 27/3604; A61L 27/54; A61L 2430/34; A61L 2430/40

(57) ABSTRACT

The present invention relates to a tissue polyhedron suspension, wherein said tissue fragments are obtainable by cutting donor tissue using a defined device. Furthermore, the invention discloses a device for preparing the suspension and various methods of using the suspension for treating condition by injecting the suspension into tissue, dripping, or pipetting, or squirting, or spraying the suspension onto tissue. The present invention relates to a device and method for tissue-structure isolation and a resulting suspension of polyhedron-shaped tissue fragments, exhibiting the three-dimensional (3D) tissue super-structures of the original structures. These tissue fragments are tissue micro-transplants such as polyhedron-shaped tissue micro-transplants, obtainable by non-destructive medical precision-cutting donor tissue using the described device. Furthermore, the invention discloses a device for preparing the suspension and various methods of using the suspension for treating condition by injecting the suspension into tissue, dripping, or pipetting, or squirting, or spraying the suspension onto tissue in need of repair or regeneration.

### TECHNICAL FIELD

The present invention relates to tissue-structure isolation and a resulting suspension of tissue fragments, exhibiting the three-dimensional (3D) tissue super-structures of the original structures, wherein said tissue fragments are tissue micro-transplants such as polyhedron-shaped tissue micro-transplants, obtainable by non-destructive medical precision-cutting donor tissue using a here defined device. Furthermore, the invention discloses a device for preparing the suspension and various methods of using the suspension for treating condition by injecting the suspension into tissue, dripping, or pipetting, or squirting, or spraying the suspension onto tissue in need of repair or regeneration.

### BACKGROUND

Chronic and acute tissue injuries and age-related degeneration in some cases are not treatable well enough by conventional surgery. The field of regenerative medicine seeks to develop innovative approaches of treating acute tissue injury, and/or chronic pathologic tissue conditions, and/or degenerative tissue damage-by regenerative approaches involving viable biologic cells, preferably human cells and human adult stem cells and preferably autologous cells, stem cells and progenitor cells. Acute and chronic and degenerative tissue damage situations often include more than one tissue typical cell type and more than one tissue layer. Injuries often concern a three-dimensional tissue structure, where various cell types of the original tissue are involved, including the regenerative progenitor and adult stem cells of that tissue and their biological “niche”—the structures that involve the biomatrix and other supportive substances and factors around these cells. Such tissues include, e.g., hair root tissue, mesenchymal tissue (including from dermis, bone marrow and fat), skin tissue, cartilage, bone, and muscle and heart muscle).

Due to the complexity of such tissue damage or degeneration, medical treatments present a medical challenge. The prior art describes methods for preparing a solution of single cells obtained from such tissues, involving a multi-step treatment of a tissue sample with various chemicals or enzymes (such as trypsin or collagenase) and results in a sprayable solution of individualized cells, typically only the dominant cell type. In such enzymatic procedures, the yielding cell types from a tissue depend on the enzymes used and often not all cell types are isolated. Moreover, due to the required washing steps, typically the biomatrix structure and other supportive substances and factors around the cells are lost. However, the effort of obtaining a transplantable solution of tissue cells by a complex and lengthy multi-step process indicates that the prior art methods are neither suitable for several tissues nor suitable in situations where a low-cost treatment is desirable (such as in cosmetic surgery), or a multitude of patient treatments simultaneously are desirable (such as in a disaster medicine when limited clinical resources are available and many patients need to be treated at once).

The inventor was interested in providing cells of a tissue therapeutically, for organ repair and regeneration by grafting (transplantation), using, e.g., injection subdermal, intramuscular, into organs such as liver or kidney or pancreas, or surface application such as onto skin by dripping. However, containing a cell type composition in the suspension approaching the original cell type composition in the target tissue. Tissues typically include the organ-typical cells but also vascular cells, and importantly a tissue-typical biomatrix with regenerative factors around the various cells. Providing this matrix with all factors involved together with the cells was another task. An example is given for hair roots where the hair root bulge contains—in addition to vascular cells and biomatrix—various cell types and stem cells and progenitor cells of various origin, that only together can grow a hair. If a hair bulge is explanted, manually cut into small pieces and then auto-grafted (transplanted), a hair bulge fragment can grow a new hair, but only as long as all components from the original bulge are present. Hence, in contrast to enzymatic methods representing the state of the art, the invention seeked to include all cell types and matrix components from a tissue. A similar approach can be deducted for liver tissue transplants and many more approaches—that today typically are provided as single cell transplantation, e.g. involving injection of hepatocytes or involving dispersion of such cells, but without the supportive cells and the surrounding matrix forming the cell nice.

Thus, there was a need to provide a device and a method for replacing tissue in patients in need of tissue regeneration or repair, and it was a requirement that the treatment biologically more complete, is more readily available, and more efficient than the treatments of the prior art. Preserving the 3D tissue super-structures of the original tissue. Specially, a method for the isolation/provision of the cells was desirable, that can be applied to many kind of tissues, instead of only one layer of one tissue in the prior art. Moreover, an approach that can be easily automated was of interest.

The above need is met by the suspension of the present invention, which comprises tissue fragments, which are tissue micro-transplants such as polyhedron-shaped tissue micro-transplants, rather than single cells, and by using a device that allows preparing the suspension in a one-step method. As the method involves no enzymatic digestion to single cells and leaves the content in the tissue micro-transplants intact, all cells from various origin in the tissue and biomatrix and factor, that were present within the tissue prior to the procedure and using the device, can be offered therapeutically. In the case of skin, epidermal cells, including progenitor cells and adult stem cells, and dermal cells, including progenitor cells and adult stem cells, as well as the skin biomatrix with all its associated factors and growth factors can be offered. From the surgical method of mesh-grafting it is known clinically, that a grafting of larger tissue fragments leads to an outgrowth of all various tissue cells into the transplanted area, and to full organ recovery. From mesh-grafting it is also known that the smaller the fragments are the better the surgical take rates and the smaller the distances between the fragments the better the cosmetic result.

Larger tissue transplants from the state of the art, e.g. mesh-grafting of skin, can be associated with bad take rates as the center of the tissues is not well served in terms of nutrition and oxygenation. Smaller micro-transplant enjoy more oxygen and blood plasma supply in their environment when transplanted. In order to provide all cells and matrix factors, they need to be large enough to contain all these factors.

The invention at hand takes this information into tissue fragments that are large enough to contain all content but are small enough to improve nutrition and oxygen support in the smaller center. Consequently, one micro-transplant contains all tissue super-structures of the original tissue, as never dissociated also in the original 3D tissue super-structure configuration. The resulting micro-transplants (polyhedrons) have a polyhedron-like outer shape. The described polyhedron-like shape occurs as a consequence of random sharp precision cuts-using micro surgical blades (FIGS. 7-10). The inventor found that because of their size, the micro transplants of the invention can be easier applied surgically, e.g. by pipetting or spraying.

As demonstrated in the Examples illustrating the present invention, the suspension of the present application is drippable, pipettable, and/or sprayable onto tissue surfaces; or can be applied by injection.

### SUMMARY

Accordingly, the present invention provides in a first aspect a suspension of tissue fragments, which are micro-transplants such as polyhedron-shaped micro-transplants, wherein said tissue fragments are obtainable by processing donor tissue using rotating blades, e.g. a device as described herein comprising a polyhedron generating tool, and wherein the tissue fragments such as polyhedron-shaped tissue micro-transplants in the suspension preferably comprise at least 100 cells.

In one embodiment, the suspension of tissue fragments of the invention is a medicinal suspension of polyhedron-shaped tissue micro-transplants, also termed “tissue polyhedron suspension”, which is preferably sterile. In one embodiment, the tissue fragments are polyhedron-shaped micro-transplants, i.e., tissue fragments in the form of polyhedrons, which are for micro-transplantation, which are preferably medicinal sterile. In one embodiment, the polyhedron-shaped tissue micro-transplants of the tissue polyhedron suspension of the invention comprise adult stem cells, stem cell supporting cells, organ-typical parenchymal cells, supporting biomatrix and cell supporting factors/mediators/cytokines/hormones. In one embodiment, the tissue polyhedron suspension and/or the polyhedron-shaped tissue micro-transplants of the tissue polyhedron suspension of the invention comprise all cells or cell types of the donor tissue.

In one embodiment, the tissue fragments of the tissue polyhedron suspension of the present invention such as the polyhedron-shaped tissue micro-transplants of the tissue polyhedron suspension have been obtained from a donor tissue, e.g., tissue explants provided from a donor. Preferably, the suspension comprises all cell types of said donor tissue and the biomatrix with associated regenerative factors surrounding the cells.

In one embodiment, the tissue polyhedron suspension of the present invention is a suspension in a physiologically acceptable solution. The physiologically acceptable solution is preferably selected from the group consisting of phosphate buffered saline (PBS), a physiological solution of sodium and potassium, a physiological solution of saline, a physiological solution of sodium, potassium magnesium and calcium, Ringer's solution, Ringer's lactate solution, physiological Hartmann solution, and a physiological solution comprising HEPES buffer.

In one embodiment, the tissue polyhedron suspension of the present invention is sprayable by a spray device and/or drippable by a dripping device. In one embodiment, the suspension can be used for treating a condition by injecting the suspension into tissue, dripping, or pipetting, or squirting, or spraying the suspension onto tissue in need of repair or regeneration.

In one embodiment, the tissue polyhedron suspension of the present invention has not been prepared by a method comprising a step of chemical or enzymatic disruption of tissue. Preferably, the tissue polyhedron suspension does not contain traces of an enzyme or chemical compound. The term “enzyme” as used herein preferably refers to an enzyme which is selected from the group consisting of trypsin, dispase, preferably dispase I or dispase II, collagenase, thermolysin, pronase, hyaluronidase, elastase, papain, proteinase K, and pancreatin. The term “chemical compound” as used herein preferably refers to a chelating agent. The chelating agent is preferably selected from the group consisting of ethylenediaminetetraacetic acid (EDTA), ethylene glycol-bis(β-aminoethyl ether)-N,N,N′,N′-tetraacetic acid (EGTA), Fura-2, Fura-2AM, Indo-1 and 1,2-bis(o-aminophenoxy) ethane-N,N,N′,N′-tetraacetic acid (BAPTA).

In one embodiment, the tissue polyhedron suspension of the present invention is obtainable by cutting donor tissue. Preferably, the donor tissue is comprised in a biological sample comprising said tissue. The term “donor tissue”, “tissue explant” and similar terms as used herein is or comprises hair-root tissue, mesenchymal tissue (including dermis and fat, skin tissue, cartilage, bone, muscle, heart muscle), epithelial tissue (including epidermal skin tissue), endothelial tissue (including vessels), ectodermal tissue, mesodermal tissue, endodermal tissue and, optionally, bio-matrix components including proteins and proteoglycans, hormones, cytokines, mediators or growth factors.

In a second aspect, the present invention relates to a device for preparation of a tissue polyhedron suspension from a biological sample comprising donor tissue, the device comprising:
a chamber (“procurement chamber”) for receiving and processing the donor tissue, the procurement chamber having a first end and a second end,
   wherein the first end comprises a first opening of the procurement chamber and, optionally, a means for closing the first opening, and wherein the second end is a closed end or comprises a second opening of the procurement chamber which is in fluid communication with a means for reversibly closing the second opening, wherein said means for reversibly closing the second opening preferably comprises a Luer-lock cap, a Luer-lock stopcock, Luer-lock connectors or a tube clamping segment; and
a polyhedron generating tool that is reversibly insertable into the procurement chamber through the open first end, the polyhedron generating tool comprising at least one upper cutting blade and at least one lower cutting blade rotatably mounted on an agitating element and spaced apart by at least one spacer element, the spacer element defining a space between the upper and lower cutting blade allowing for passage and cutting of the tissue in the procurement chamber, the agitating element further comprising an engagement portion that is adapted to be engaged by a drive mechanism such as a hand operated drive mechanism, e.g., is or comprises a gear, or a motor, wherein the polyhedron generating tool is configured to allow for the generation of fluidic turbulences and fluid movements of the suspension fluid during rotation of the upper and lower cutting blade, therefore the cutting side of the upper and lower cutting blade is positioned against the resultant direction of rotation of the biological sample in the fluid suspension.

In a third aspect, the present invention relates to a device for preparation, such as non-destructive preparation, of a suspension of polyhedron-shaped tissue micro-transplants (“tissue polyhedron suspension”) from a biological sample comprising donor tissue, preferably using disposable precision micro-surgery scalpel blades such as rotating disposable precision micro-surgery scalpel blades, the device comprising:
a chamber (“procurement chamber”) for receiving and processing the donor tissue, the procurement chamber comprising
   a first opening for providing tissue explants into the procurement chamber,
   a second opening at a height that allows removing tissue polyhedron suspension from the procurement chamber, and
   a third opening at a height that allows adding liquid into the procurement chamber; and
a polyhedron generating tool that is reversibly insertable into the procurement chamber through the first opening, the polyhedron generating tool comprising at least one upper blade, such as a micro-surgery scalpel blade, and at least one lower blade, such as a micro-surgery scalpel blade, rotatably mounted on an agitating element and spaced apart by at least one spacer element, the spacer element defining a space between the upper and lower blade allowing for passage and layer-shaving, such as precision micro-surgery standard sharp non-destructive layer-shaving, of the tissue in the procurement chamber into polyhedron-shaped tissue micro-transplants, the agitating element further comprising an engagement portion that is adapted to be engaged by a drive mechanism, such as a hand operated drive mechanism or a drive mechanism that is or comprises a motor, wherein the polyhedron generating tool is configured to allow for the generation of fluidic turbulences and fluid movements of the suspension fluid during rotation of the upper and lower blade, for a rotating layer-shaving, therefore the cutting side of the upper and lower blade is positioned against the resultant direction of rotation of the biological sample in the fluid suspension.

In a fourth aspect, the invention relates to an automated medical device for non-destructive preparation, i.e., tissue-structure isolation, of a sterile suspension of medical tissue fragments (micro-transplants) that are polyhedron shaped and exhibit the three-dimensional (3D) tissue super-structures of the original structures (“tissue polyhedron suspension”), using disposable precision micro-surgery scalpel blades, from a biological sample comprising donor tissue, yielding-in contrast to tissue chopping or mincing-viable regenerative cells in the inside of the polyhedrons maintaining the tissue super-structure with their original 3D tissue cell-cell and cell-niche structure junctions with all parenchymal and non-parenchymal cells and the connective tissue matrix with growth factors preserved in the original 3D tissue configuration, the device comprising:
a chamber (“procurement chamber”) for receiving and processing the donor tissue, the procurement chamber comprising
   a first opening for providing tissue explants into the procurement chamber,
   a second opening at a height that allows removing tissue polyhedron suspension from the procurement chamber, and
a third opening at a height that allows adding liquid into the procurement chamber; and a polyhedron generating tool that is reversibly insertable into the procurement chamber through the first opening, the polyhedron generating tool comprising at least one upper micro-surgery disposable scalpel blade and at least one lower micro-surgery disposable scalpel blade rotatably mounted on an agitating element and spaced apart by at least one spacer element, the spacer element defining a space between the upper and lower micro-surgery scalpel blade allowing for passage and precision micro-surgery standard sharp non-destructive layer-shaving of the tissue in the procurement chamber into medicinal sterile polyhedron-shaped tissue micro-transplants that exhibit under the outer surface of cut cells the intact and viable cells from the original tissue in a way maintaining their original 3D tissue superstructure and tissue cell-cell and cell-niche structure with all parenchymal and non-parenchymal cells and the connective tissue matrix with growth factors preserved intact and able to survive in cell culture, show cell outgrowth in vitro, cell divisions and regenerative actions including cell divisions and migration in cell culture, the agitating element further comprising an engagement portion that is adapted to be engaged by a drive mechanism such as a hand operated drive mechanism, e.g., is or comprises a gear, or a motor, wherein the polyhedron generating tool is configured to allow for the generation of fluidic turbulences and fluid movements of the suspension fluid during rotation of the upper and lower micro-surgery disposable scalpel blade, for a rotating layer-shaving, therefore the cutting side of the upper and lower micro-surgery scalpel blade is positioned against the resultant direction of rotation of the biological sample in the fluid suspension, whereas the explants pass vertically through the blade action plane and re-enter the process multiple times but each time in a changed vertical orientation, resulting in precision-dividing larger organ explants into micro-transplants for regenerative transplantation, micro-transplants with viable cells in the inside that feature multiple straight-cut outer flat surfaces in a random distribution and are thereby polyhedron shaped and because of the non-destructive nature feature all cell populations in the natural tissue super-structure composition preserving the Levels of Structure in tissues (cellular level, biomatrix level microvascular level, repetitive microvascular levels) and the tissue adult stem cell niches with biomatrix in the original 3D tissue configuration.

In one embodiment, the disposable precision micro-surgery scalpel blades of the device of the invention are arranged as an assembly of blades, such as described herein. Thus, in one embodiment, the polyhedron generating tool comprises an assembly of blades as described herein. In one embodiment, the agitating element of the device of the invention is a rotating agitating element. In one embodiment, the engagement portion is at the end of the rod or shaft of the agitating element of the device of the invention, as described herein. In one embodiment of the polyhedron generating tool of the device of the invention, the cutting side of the upper and lower blades, e.g., micro-surgery scalpel blades, is position against the resultant direction of rotation of the biological sample in the fluid suspension at an angle of between 1° and 15°.

In one embodiment, the device of the invention is a medical device, e.g., an automated medical device. In one embodiment, the device of the invention is for non-destructive preparation of a suspension of, optionally polyhedron-shaped, tissue micro-transplants using blades such as disposable sharp micro-surgery scalpel blades, from a biological sample comprising donor tissue, yielding-in contrast to tissue chopping or mincing-viable regenerative cells in the inside of the polyhedrons maintaining the tissue super-structure with their original three-dimensional (3D) tissue cell-niche structure with all parenchymal and non-parenchymal cells and the connective tissue matrix with growth factors preserved in the original 3D tissue configuration, as described in herein. In one embodiment, the polyhedron-shaped micro-transplants of the invention are obtained by non-destructive medical precision-cutting of donor tissue using the device of the invention. In one embodiment, the micro-transplants of the invention exhibit under the outer surface of cut cells the intact and viable cells from the original tissue in a way maintaining their original three-dimensional 3D tissue superstructure and tissue cell-cell and cell-niche structure with all parenchymal and non-parenchymal cells and the connective tissue matrix with growth factors preserved intact and able to survive in cell culture, show cell outgrowth in vitro, cell divisions and regenerative actions including cell divisions and migration in cell culture. In one embodiment, the preparation such as the non-destructive preparation is tissue-structure isolation. In one embodiment, the configuration of the device, in particular of the polyhedron generating tool, is such that a medicinal sterile suspension comprising medicinal sterile polyhedron-shaped tissue micro-transplants can be produced.

In one embodiment, the polyhedron-shaped tissue fragments micro-transplants are medical tissue fragments that are polyhedron shaped and exhibit the three-dimensional (3D) tissue super-structure of the original structures they are derived from. In one embodiment, the polyhedron-shaped tissue micro-transplants exhibit under the outer surface of cut cells the intact and viable cells from the original tissue in a way maintaining their original three-dimensional (3D) tissue superstructure and tissue cell-niche structure with all parenchymal and non-parenchymal cells and the connective tissue matrix with growth factors preserved intact and able to survive in cell culture, show cell outgrowth, cell divisions and regenerative actions including cell divisions and migration in cell culture.

In one embodiment of the device of the invention and of the method of the invention, the explants pass vertically through the blade action plane and re-enter the process multiple times but each time in a changed vertical orientation, resulting in precision-dividing larger organ explants into micro-transplants for regenerative transplantation, micro-transplants with viable cells in the inside that feature multiple straight-cut outer flat surfaces in a random distribution and are thereby polyhedron shaped and because of the non-destructive nature feature all cell populations in the natural tissue super-structure composition preserving the Levels of Structure in tissues (cellular level, biomatrix level microvascular level, repetitive microvascular levels) and the tissue adult stem cell niches with biomatrix in the original 3D tissue configuration.

“Polyhedron generating tool” or simply “tool” refers to a tool comprising at least one upper and at least one lower scalpel blade rotatably mounted on an agitating element and spaced apart by at least one spacer element defining a space between the upper and lower scalpel blade. This allows for passage and layer-shaving, such as precision micro-surgery standard sharp non-destructive layer-shaving, of tissue when rotating the tool. In one embodiment, the at least one upper blade and at least one lower blade protrude in radial direction from the agitating element when the tool is seen in top view. In one embodiment, the polyhedron generating tool of the device of the invention comprises one or more blades as described herein arranged as a blade assembly. In one embodiment, the cutting blade assembly of the polyhedron generating tool of the device of the invention uses disposable surgical and/or disposable micro-surgery scalpel blades such as precision micro-surgery scalpel blades. Thus, in one embodiment, the polyhedron generating tool comprises at least 2, 3, 4, 5, 6, 7, 8, 9, 10, 11, 12, 13, 14, 15, 16, 17, 18, 19, or 20 blades. In one embodiment, the polyhedron generating tool comprises 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, or 20 or more blades. In one embodiment, the polyhedron generating tool comprises up 20, up to 19, up to 18, up to 17, up to 16, up to 15, up to 14, up to 13, up to 12, up to 11, up to 10, up to 9, up to 8, up to 7, up to 6, up to 5, up to 4, up to 3, or up to 2 blades. In one embodiment, the polyhedron generating tool comprises 8 blades. In one embodiment, the polyhedron generating tool comprises 4 blades. In one embodiment, the polyhedron generating tool comprises 6 blades.

In one embodiment, the agitating element of the polyhedron generating tool of the device of the invention comprises a rod or shaft. The agitating element may comprise means for attaching the blade assembly of the polyhedron generating tool. In one embodiment, the blades are mounted directly on the agitating element. In one embodiment, the blades are mounted indirectly on the agitating element, e.g., by being mounted on or attached to a holding element, which is then mounted on the agitating element. In one embodiment, the holding element comprises a body with a cylindrical form or a cuboid form, for example wherein two of the faces of the cuboid are squares and four other faces are rectangular in shape, preferably wherein the edges of the cuboid are rounded. In one embodiment, the blades protrude from the holding element. In one embodiment, the body of the holding element is monolithic, i.e., made from one piece of material, wherein the body comprises means for attaching blades, such as groves, into which a part of a blade can be inserted and be fixed, wherein the means are at different heights, when the body is seen in side view. In this embodiment, the blades protruding from the holding element are spaced apart, i.e., the holding element functions as a spacer element spacing apart the blades. In one embodiment, the body comprises several parts, such as a body that is segmented wherein each blade is inserted between two segments respectively and the blades are attached to one or both segments they are inserted in between and/or fixed. In this embodiment, one or more of the segments may function as spacer elements spacing apart the blades of the polyhedron generating tool, i.e., the holding element comprises segments spacing apart the blades. Fixation or attachment of blades to the agitating element can be accomplished using any fixation means known in the art for attaching or fixating a blade in a medical-technical context, e.g., biocompatible or medical-technical glue, screws and so on. The blades may also be fixed between the spacing elements by press fitting. For example, the body of the holding element may comprise a stack of segments, wherein blades are press fit between segments, wherein the segments are hold within the stack by e.g., using clamps, screws or other means that can be used to hold the stack of segments together. In another example, the body of the holding element may comprise a stack of segments held together with glue or another fixation means and the blades are inserted between the segments. A segment spacing apart two blades from one another is or functions as a spacer element. It is preferred that the body of the holding element comprises a stack of segments because this embodiment allows for easy disassembly and reassembly, e.g., for cleaning purposes and for replacement of blades. Moreover, some or all blades attached to the body of the holding element may be attached such that the cutting edge and the blunt edge of a given blade are at different heights, when the tool is seen in side view. For example, in one embodiment, the blades are positioned at an angle of 0° to 45° such as 1° to 30°, preferably 1° to 15°, against the resultant direction of rotation of the biological sample in the fluid suspension. For example, the tool may comprise blades that, when the tool is seen in side view, are arranged such that their cutting edges are above their blunt edges. For example, the tool may comprise blades that, when the tool is seen in side view, are arranged such that their cutting edges are below their blunt edges. For example, the tool may comprise blades that, when the tool is seen in side view, are arranged such that their cutting edges are above their blunt edges and blades that are arranged such that their cutting edges are below their blunt edges. Optionally, the tool may comprise blades that are arranged such that, when the tool is seen in side view, their cutting edges and blunt edges are horizontal. For example, the top and bottom faces of any segment of a holding element body comprising a stack of segments, i.e., the face of the segment which when being part of a stack is in contact with the next or previous segment (if present), can be undulated such that the cutting edge and the blunt edge of a blade that is inserted between two segments fitting on top of each other within a stack of segments, wherein the top segment of said two fragments has an undulated bottom face and the bottom segment of said two fragments has an undulated top face, will not be at same height when the tool the segment stack is part of is seen in side view. The polyhedron generating tool is reversibly insertable into the procurement chamber of the device of the invention, e.g., through a first opening.

In one embodiment, the blades of the polyhedron generating tool of the invention are arranged such that when the tool is seen in top view, the blades protrude from the agitating element and/or the holding element of the tool such that the tips of a given group of blades have the largest possible distance from one another. The tool may comprise several groups of blades. For example, a first group of blades, wherein their tips have the largest possible distance from one another, and optionally a second group of blades, wherein their tips have the largest possible distance from one another, and optionally a third group of blades, wherein their tips have the largest possible distance from one another, and optionally a fourth group of blades, wherein their tips have the largest possible distance from one another, are present. That is, the polyhedron generating tool comprises at least one group of blades. Of course, more than four groups of blades may be present such as five, six, seven, eight, nine or ten groups of blades, wherein for each group, the tips have the largest possible distance from one another. The tips of the blades of one group, however, do not need to have the largest possible distance to one or all blade tips of another group. That is, largest possible distance refers to the blades of the same group. In one embodiment, a given group of blades comprises all blades that, when seen in top view of the polyhedron generating tool, are neither fully nor partly covered by other blades. For example, the first group of blades includes all blades that, in top view of the polyhedron generating tool, are not fully or partly covered by other blades. In top view of the tool, the second group of blades would then include all blades that would neither fully nor partly be covered by other blades, if all blades of the first group would be removed. The same applies to the third group if all blades of the second group would be removed, to the fourth group if all blades of the third group would be removed and so on. Alternatively or in addition, blades may be defined as belonging to the same group of blades, if, when seen in side view of the polyhedron generating tool, the blades are at the same height. A given blade may not be fully visible when being part of the tool, e.g., part of a given blade may be covered by parts of a spacer element and/or of the body of the holding element including any spacer element or any attachment means attaching the blade to another element.

As used herein, “top view” or similar terms and “side view” or similar terms refer to the orientation of the polyhedron generating tool when inserted into the device of the invention.

In one embodiment, the blades of the first group of the polyhedron generating tool of the invention are arranged such that the tips of the blades describe a (imaginary) triangle when seen in top view of the tool. In one embodiment, the blades are arranged such that when the polyhedron generating tool is seen in top view, the blades protrude in a cross-like arrangement from the agitating element and/or from the holding element. When seen from the side of the tool, the blades can be arranged in one or more planes or at one or more heights. For example, when arranged cross-like as described above, the two blades of one of the two axes of the cross may be arranged in a first plane or at a first height and the two blades of the other axis of the cross may be arranged in a second plane or at a second height. However, the blades belonging to the same group of blades may also all be at different height. For example, the tool may comprise 4 blades, which may be seen as one group of blades, and which may all be at different heights and may optionally be in cross-like arrangement, as described herein. The tool can also comprise more than one geometrical arrangement. For example, the tool may comprise a second cross-like arrangement of blades below a first cross-like arrangement of blades, wherein both arrangements are as described herein. For example, when the tool is seen in side view, a first axis of a first cross-like arrangement may be at a first height, a second axis of a first cross-like arrangement may be above or below the first axis, a first axis of a second cross-like arrangement may be below the first axis of the first cross-like arrangement and a second axis of the second cross-like arrangement may be below the second axis of the second cross-like arrangement of blades, wherein the arrangements appear cross-like when the tool is seen in top view. Likewise, the blades can be arranged in other (imaginary) geometrical forms, e.g., triangular, pentagonal, hexagonal and so on, wherein the tips of the blades are the angles of the (imaginary) geometrical form. When arranged in a (imaginary) geometrical form, all blades forming said geometrical form may be in a first plane when the tool is seen in side view or may be at different heights, and a second geometrical form, e.g., a second triangular blade arrangement, may be below or above the first geometrical form, e.g., the first triangular blade arrangement.

The blades protruding from the agitating element and/or the holding element are arranged such that the tips of the blades are away from the centre of rotation when the tool is inserted into the device. It is preferred to use blades in beveled or tapered orientation with respect to the direction of rotation of the polyhedron generating tool. Using blades in beveled or tapered orientation with respect to the direction of rotation of the tool results in movement of liquid in the desired direction and in generation of swirls or turbulences within the liquid (see FIGS. 7-9). The blades can be arranged such that when the tool is seen in side view, the cutting edge is higher than the blunt edge or vice versa, as described herein. In one embodiment, the cutting edge of all blades of a given height or of a given group, e.g., the first and/or second group, can face the same direction with respect to the direction of rotation of the tool. In another embodiment, the cutting edge of every other blade of a given height or of a given group, e.g. the first and/or second group, can face a direction that differs from the other blades of the same group with respect to the direction of rotation of the tool. Moreover, the blades of a given axis, of a given height or of a given group, e.g., the first axis or the first group, can be arranged such that, when seen from the side of the tool, the cutting edge is above the blunt edge. In another embodiment, the blades of a given axis, of a given height or of a given group, e.g., the first axis or the first group, can be arranged such that, when seen from the side of the tool, the cutting edge is below the blunt edge. In another embodiment, the blades of a given axis, e.g., the first axis, can be arranged such that, when seen from the side of the tool, the cutting edge of a first blade is above its blunt edge and the cutting edge of a second blade is below its blunt edge. Alternatively, the blades of the first axis can be in plane. The blades of another axis, e.g., of the second axis, may be arranged as described for the first axis or may be in plane. Moreover, when rotating the tool, all blades may face in the same direction. Alternatively, when rotating the tool, a number of blades may face in the opposite direction, i.e., against the rotational direction. Arranging the blades such that a number of blades faces against the rotational direction results in a tool that can be used in more than one rotational direction.

Thus, the polyhedron generating tool of the invention can comprise an even number of blades such as 4 or 8 blades in one or two cross-like arrangements (when the tool is seen in top view), wherein the blades of the first axis of the first cross-like arrangement are arranged such that (when the tool is seen in side view) the cutting edge of a first blade is higher than its blunt edge and the cutting edge of a second blade is lower than its blunt edge, and the blades of the second axis of the first cross-like arrangement are arranged such that (when the tool is seen in side view) the cutting edges of both blades are in plane, wherein the first axis is below the second axis, optionally, wherein a second cross-like arrangement is at a lower height than the first cross-like arrangement (when the tool is seen in top view), wherein the blades of the first axis of the second cross-like arrangement are arranged such that (when the tool is seen in side view) the cutting edge of a first blade is lower than its blunt edge and the cutting edge of a second blade is higher than its blunt edge, and the blades of the second axis of the first cross-like arrangement are arranged such that (when the tool is seen in side view) the cutting edges of both blades are in plane, wherein the first axis is at a lower height than the second axis. In one embodiment, (when the tool is seen in top view) the first axis of the first cross-like arrangement is congruent with the first axis of the second cross-like arrangement, and the second axis of the first cross-like arrangement is congruent with second axis of the second cross-like arrangement. In one embodiment, (when the tool is seen in side view) the cutting edges of the first blades of the first axes of the first and second cross-like arrangements are higher that their blunt edges and the cutting edges of second blades of the first axes of the first and second cross-like arrangements are lower that their blunt edges and all other blades of the axes of both cross like arrangements are as described above. In one embodiment, all blades of the tool face the same direction with respect to the direction of rotation of the tool.

In one embodiment, the polyhedron generating tool comprises means for generating of fluidic turbulences and fluidic movements of the suspension fluid during rotation, e.g., means such as a paddle-wheel, an impeller, a bucket wheel, protrusions, indentations or notches. In one embodiment, the means for generating fluidic turbulences may be attached to and/or formed from any rotating part such as the agitating element or the holding element. In one embodiment, the means are present in separate form, e.g. as an element attached above or below the blade assembly of the polyhedron generating tool.

In one embodiment, the procurement chamber of the device of the present invention comprises a first and a second end, wherein the first end comprises the first opening. In one embodiment, the first opening comprises means for reversibly closing the first opening. In one embodiment, the second opening of the procurement chamber of the device of the present invention is located near the second end of the procurement chamber. In one embodiment, the third opening of the procurement chamber of the device of the present invention is located near the first end of the procurement chamber. In one embodiment, means for reversibly closing an opening comprise a Luer-lock cap, a Luer-lock stopcock, Luer-lock connectors or a tube clamping segment.

In one embodiment, the second and/or the third opening of the procurement chamber of the device of the invention is in fluid communication with a means for reversibly closing said opening, wherein the means for reversibly closing said opening preferably comprise a Luer-lock cap, a Luer-lock stopcock, Luer-lock connectors or a tube clamping segment.

In one embodiment, the means of the device of the invention for reversibly closing the second opening are in fluid communication with one or more layers of filtration membrane, which are preferably located between the second opening of the procurement chamber and the means for closing the second opening.

In one embodiment, the means of the present invention for closing the first opening is a closing lid for the procurement chamber, and the polyhedron generating tool is arranged in the closing lid in such a way that it is rotatably mounted through a bore such as a central bore of the closing lid. For example, the lid may be attached to the procurement chamber using glue or it may be a screw-on lid or a snap-on lid. In one embodiment, the closing lid for the procurement chamber is connectable to the first end of the procurement chamber. Thus, the lid and the blades with the agitating elements may be combined in one unit or assembly that can be connected to a motor. In one embodiment, the lid, the blades, the blade assembly, the agitating element, the shaft, the liquid chamber, the tissue polyhedron suspension chamber and/or the procurement chamber of the device of the invention are disposable.

In one embodiment, the procurement chamber of the present invention has a tubular shape with an inner diameter of 1 to 15 cm, preferably of 2 to 6 cm.

In one embodiment, the procurement chamber of the present invention has a volume of 5 to 1000 ml, preferably of 50 to 250 ml.

In one embodiment, the procurement chamber of the present invention is a disposable sterile cell biology laboratory centrifugation tube, preferably of 50 to 250 ml.

In one embodiment, the procurement chamber of the present invention is a disposable sterile centrifugation tube that is modified with additional openings.

In one embodiment, the blade and lid assembly of the present invention comprises a holding bracket that allows for automated transfer of the blades into and out of a procurement chamber.

In one embodiment, the procurement chamber of the present invention comprises a holding bracket that allows for automated transfer of the procurement chamber into and out of a centrifuge.

In one embodiment, the procurement chamber of the present invention comprises at least one additional opening and a means for closing the additional opening, wherein the opening is preferably located in the side wall of the procurement chamber, preferably at a height that allows removing supernatant of centrifugation.

In one embodiment, the procurement chamber of the invention comprises a second opening and a third opening, wherein the second opening is at a height that allows removing tissue polyhedron suspension produced, e.g., during operation of the device, and the third opening is at a height that allows adding liquid, e.g., during operation of the device. Optionally, the second and/or the third opening is in fluid communication with a means for reversibly closing said opening, wherein said means for reversibly closing said opening preferably comprise a Luer-lock cap, a Luer-lock stopcock, Luer-lock connectors or a tube clamping segment.

In one embodiment, the device of the invention comprises a liquid chamber configured to receive liquid and to provide the liquid into the procurement chamber. In one embodiment, the liquid chamber is removably connected to the procurement chamber. Preferably, the liquid chamber is configured to provide liquid into the procurement chamber during operation of the device. Preferably, the liquid chamber is in fluid communication with the third opening of the procurement chamber. The liquid chamber of the invention may be a vessel, which is optionally disposable and/or sterile. The liquid chamber of the invention may comprise or may be a syringe such as a sterile medical syringe such as a disposable syringe or a vessel, which is optionally disposable and/or sterile. In one embodiment, the procurement chamber is configured to accept the liquid chamber. In one embodiment, the liquid provided by the liquid chamber into the procurement chamber is a physiologically acceptable solution as described herein. In one embodiment, the liquid provided by the liquid chamber into the procurement chamber comprises one or more buffer solutions as described herein. In one embodiment, liquid can be added into and/or removed from the liquid chamber of the device using one or more pumps such as roller tube pumps, fingerprint tube pumps, piston pumps and/or plunger pumps. For example, the liquid chamber of the device comprises a sterile vessel that is optionally disposable and liquid can be added thereto and/or removed therefrom using one or more pumps such as roller tube pumps, fingerprint tube pumps, piston pumps and/or plunger pumps.

Thus, in one embodiment, the device of the invention comprises one or more pumps configured to add liquid into the liquid chamber, wherein the one or more pumps preferably are selected from the group consisting of roller tube pumps, fingerprint tube pumps, piston pumps and plunger pumps. In one embodiment, the device of the invention comprises one or more pumps configured to remove liquid from the liquid chamber, e.g., into the procurement chamber, wherein the one or more pumps preferably are selected from the group consisting of roller tube pumps, fingerprint tube pumps, piston pumps and plunger pumps. Of course, the pump adding liquid into the liquid chamber and the pump removing liquid from the liquid chamber may be the same or may be different. The one or more pumps may be hand actuated pumps or may be operated by automation means, e.g, as described herein.

In one embodiment, the device of the invention comprises a chamber (“tissue polyhedron suspension chamber”) configured to accept tissue polyhedron suspension, and optionally to remove the tissue polyhedron suspension from the procurement chamber into the tissue polyhedron suspension chamber. In one embodiment, the tissue polyhedron suspension chamber is removably connected to the procurement chamber. Preferably, the tissue polyhedron suspension chamber is configured to remove tissue polyhedron suspension from the procurement chamber during operation of the device. Preferably, the tissue polyhedron suspension chamber is in fluid communication with the third opening of the procurement chamber. The tissue polyhedron suspension chamber of the invention may comprise or may be a syringe such as a sterile medical syringe such as a disposable syringe, or a vessel, which is optionally disposable and/or sterile. In one embodiment, the procurement chamber is configured to accept the tissue polyhedron suspension chamber. In one embodiment, the tissue polyhedron suspension chamber is configured to be accepted by a tissue polyhedron suspension spray device and/or a tissue polyhedron suspension dripping device. In one embodiment, the tissue polyhedron suspension chamber is configured to be accepted by a tissue polyhedron suspension spray deposition or drip deposition or injection deposition device. In one embodiment, liquid, e.g., tissue polyhedron suspension, can be added into and/or removed from the tissue polyhedron suspension chamber of the device using one or more pumps such as roller tube pumps, fingerprint tube pumps, piston pumps and/or plunger pumps. For example, the tissue polyhedron suspension chamber of the device comprises a sterile vessel that is optionally disposable and liquid, e.g., tissue polyhedron suspension, can be added thereto and/or removed therefrom using one or more pumps such as roller tube pumps, fingerprint tube pumps, piston pumps and/or plunger pumps.

Thus, in one embodiment, the device of the invention comprises one or more pumps configured to add liquid, e.g., tissue polyhedron suspension, into the tissue polyhedron suspension chamber, e.g., from the procurement chamber, wherein the one or more pumps preferably are selected from the group consisting of roller tube pumps, fingerprint tube pumps, piston pumps and plunger pumps. In one embodiment, the device of the invention comprises one or more pumps configured to remove liquid, e.g., tissue polyhedron suspension, from the tissue polyhedron suspension chamber, wherein the one or more pumps preferably are selected from the group consisting of roller tube pumps, fingerprint tube pumps, piston pumps and plunger pumps. Of course, the pump adding liquid into the tissue polyhedron suspension chamber and the pump removing liquid from the tissue polyhedron suspension chamber may be the same or may be different. The one or more pumps may be hand actuated pumps or may be operated by automation means, e.g., as described herein.

In one embodiment, the device of the invention comprises one or more pumps configured to add liquid into the procurement chamber, wherein the one or more pumps preferably are selected from the group consisting of roller tube pumps, fingerprint tube pumps, piston pumps and plunger pumps. In one embodiment, the device of the invention comprises one or more pumps configured to remove liquid from the procurement chamber, wherein the one or more pumps preferably are selected from the group consisting of roller tube pumps, fingerprint tube pumps, piston pumps and plunger pumps. Of course, the pump adding liquid into the procurement chamber and the pump removing liquid from the procurement chamber may be the same or may be different. The one or more pumps may be hand actuated pumps or may be operated by automation means, e.g., as described herein.

When using one or more pumps, the one or more pumps can be connected to the liquid chamber and/or the procurement chamber and/or the tissue polyhedron suspension chamber using tubes such as medical tubes. It will be obvious to the skilled person how to connect the various parts of the device of the present invention to one another to ensure medical, preferably sterile, conditions. Ensuring sterility may, for example, comprise the use of sterile disposable tubes and/or respective connectors connecting the tubes and pumps.

Vessels that may be used as the liquid chamber and/or the tissue polyhedron suspension chamber or be comprised in the liquid chamber and/or in the tissue polyhedron suspension chamber may be selected from the group consisting of glass vessels, glass tubes, plastic tubes, bottles, syringes, tubes, but are not limited thereto. The skilled person will readily understand that any vessel that can be sterilized or can be provided in sterile condition can be used. Preferably, the vessel is sterile and optionally medical.

In one embodiment, the device of the invention comprises means for recording and providing process parameters such as time, rpm, pump action, for patient specific documentation and/or quality management system (QMS) information. Said means can be detectors and/or probes. Any detector, probe or other means capable of recording and providing process parameters such as time, rpm, pump action, for patient specific documentation and QMS information can be used. In one embodiment, the device and/or one or more of its chambers such as the procurement chamber and/or the tissue polyhedron suspension chamber comprise means for assigning the device and/or the one or more of its chambers to a specific subject such as a specific patient, e.g., a tag such as a Radio-Frequency Identification (RFID) tag, a Near Field Communication (NFC) chip, a Quick Response (QR) code including micro QR code, secure QR code, iQR code, frame QR code, a bar code, a matrix bar code, a numerical code or another machine-readable optical label. For example, the means for assigning the device and/or the one or more of its chambers to a specific subject may contain or provide data for a locator, identifier, or tracker that points to a website, database or application, an inventory number or other data useful for assigning the device and/or the one or more of its chambers to a specific subject.

In one embodiment, the device of the invention is configured such that closed system operation for clean operation under a QMS is enabled. For example, parts removably connected to the device such as the liquid chamber and the tissue polyhedron suspension chamber may be connected using sterile Luer-lock connectors, spike ports, needle ports and other means for sterile injection of material.

In one embodiment, the device of the invention is disposable and/or for one-way use. In one embodiment, the device of the invention is sterilizable and/or for multiple use.

In a fifth aspect, the present invention relates to a drive unit comprising a drive mechanism, wherein the drive mechanism is configured to engage the engagement portion of the agitating element of the device of the invention. Thus, the drive unit is configured to rotate the polyhedron generating tool of the invention through the drive mechanism. In one embodiment, the drive unit of the invention is configured to accept and/or hold the device of the invention.

In one embodiment, the drive mechanism is or comprises a motor, e.g., an electrical motor. The advantage of using a motor is the possibility to apply a constant and/or easily controllable force independently of the physical strength of the user.

In one embodiment, the drive mechanism is a hand operated drive mechanism, e.g., is or comprises a gear such as a gear for inducing rotation. For example, the rotation may be spring induced or rip-line induced or induced using a crank handle. Thus, the drive unit may comprise a spring, rip-line or crank handle for inducing rotation. The drive unit of the present invention comprising a hand operated drive mechanism has the advantage that it can be used independently of power supply and/or batteries. This is advantageous in several use cases, e.g., disaster medicine or field medicine. Advantageously, and as is the case for all embodiments of the present invention, no enzymes are needed and, therefore, it is also not required to keep the tissue polyhedron suspension at a temperature that would allow enzymes to be active, e.g., 37° C. In one embodiment, the drive unit of the invention is configured to accept and/or hold the device of the invention.

In one embodiment, the drive mechanism is configured to engage the engagement portion of the agitating element of the device of the invention, wherein the drive unit comprises automation means for automated addition of liquid from the liquid chamber into the procurement chamber of the device. In one embodiment, the drive unit of the invention comprises a drive mechanism, wherein the drive mechanism is configured to engage the engagement portion of the agitating element of the device of the invention, wherein the drive unit is configured to accept the device, and wherein the drive unit comprises automation means for automated addition of liquid from the liquid chamber into the procurement chamber of the device. In one embodiment, the device further comprises a tissue polyhedron suspension chamber and the drive unit preferably comprises automation means for automated removal of tissue polyhedron suspension from the procurement chamber into the tissue polyhedron suspension chamber. Thus, in one embodiment, the drive unit of the invention comprises a drive mechanism, wherein the drive mechanism is configured to engage the engagement portion of the agitating element of the device of the invention, wherein the drive unit is configured to accept the device, and wherein the drive unit comprises automation means for automated removal of tissue suspension from the procurement chamber of the device into the tissue polyhedron suspension chamber. In one embodiment, the liquid chamber and/or the tissue polyhedron suspension chamber of the device are syringes as described herein and the automation means are configured to move the piston of the syringe(s). In one embodiment, the automation means are configured to operate one or more pumps to add and/or remove liquid from the liquid chamber and/or the tissue polyhedron suspension chamber and/or the procurement chamber. In one embodiment, the one or more pumps are selected from the group consisting of roller tube pumps, fingerprint tube pumps, piston pumps and/or plunger pumps.

Automation approaches may aim to enable a computerized use where the user opens a sterile disposable kit, attaches this to the non-sterile reusable part of the device, and initiates a procedure. Then, the user sets in the tissue sample and the device performs changes of liquids, performs the cutting, then a suction of resulting suspension is performed into a therapeutic vessel (e.g. a syringe for spray deposition through a cell sprayer or another vessel as described herein) and offer this vessel to the operator, ready for use.

In one embodiment, the drive mechanism such as the motor or hand operated drive mechanism is configured to engage the engagement portion of the agitating element of the device of the invention comprising a tissue polyhedron suspension chamber, wherein the drive unit comprises automation means for automated removal of tissue polyhedron suspension from the procurement chamber of the device into the tissue polyhedron suspension chamber. In one embodiment, the device further comprises a liquid chamber and the drive unit preferably comprises automation means for automated addition of liquid from the liquid chamber into the procurement chamber. In one embodiment, the liquid chamber and/or the tissue polyhedron suspension chamber of the device are syringes as described herein and the automation means are configured to move the piston of the syringe(s). In one embodiment, the automation means are configured to operate one or more pumps to add and/or remove liquid from the liquid chamber and/or the tissue polyhedron suspension chamber and/or the procurement chamber. In one embodiment, the one or more pumps are selected from the group consisting of roller tube pumps, fingerprint tube pumps, piston pumps and/or plunger pumps.

In one embodiment, the drive unit of the invention comprises operation control means configured to control acceleration, deceleration, velocity and/or vertical movement of the agitating element when the engaging portion is engaged by the drive mechanism. In one embodiment, the operation control means are one or more electronic boards connected to an external control panel that is configured to be controlled from the outside of the drive unit and interfaced with the drive mechanism. In one embodiment, the drive unit comprises a detection unit, e.g., electronically connected to the drive mechanism and interfaced with the one or more electronic boards, wherein the detection unit is configured to detect at least one parameter of the tissue polyhedron suspension selected from viscosity, cell or fragment number, cell or fragment density, cell or fragment size, liquid osmolarity, liquid oncotic pressure, optochemical probes, temperature and pH. Of course, the detection unit may also be configured to detect more than one parameter.

In a sixth aspect, the present invention relates to a method of preparing a tissue polyhedron suspension.

The method preferably comprises a step (a) of adding a biological sample comprising donor tissue to the procurement chamber of the device of the present invention and, optionally, of adding a physiologically acceptable solution; and a step (b) of operating the device under conditions that result in a suspension comprising tissue fragments such as polyhedron-shaped tissue micro-transplants obtained from the donor tissue. Preferably, the tissue fragments of the suspension comprise at least 100 cells.

In one embodiment, the method of the present invention comprises the additional steps (c) of centrifuging the suspension resulting from step (b) to produce a sediment of tissue fragments such as polyhedron-shaped tissue micro-transplants and a supernatant; (d) of removing the supernatant from the sediment of tissue fragments of step (c); and (e) of suspending the sediment in a physiologically acceptable solution, thereby producing a suspension comprising tissue fragments such as polyhedron-shaped tissue micro-transplants. Step (b) may be performed using the drive unit of the invention.

In one embodiment, rotation of the blades of the device of the invention, i.e., blade spinning, is performed at a rotational speed of from 500 to 100,000 rpm (revolutions per minute) such as from 500 to 50,000 rpm, 1,000 to 50,000 rpm, 5,000 to 50,000 rpm, 10,000 to 40,000 rpm, or 20,000 to 30,000 rpm. s

In one embodiment, the device comprises a liquid chamber configured to receive liquid and to provide the liquid into the procurement chamber, and wherein the method comprises adding liquid from the liquid chamber into the procurement chamber before or during step (b). In one embodiment, liquid is added to adapt a parameter of the tissue polyhedron suspension in the procurement chamber selected from the group consisting of viscosity, cell and fragment density, cell and fragment size, liquid osmolarity, liquid oncotic pressure, optochemical probes, temperature, and pH of the suspension. Preferably, the liquid added using the liquid chamber is a physiologically acceptable solution.

In one embodiment, the device comprises a tissue polyhedron suspension chamber configured to accept tissue polyhedron suspension, and optionally to remove tissue polyhedron suspension from the procurement chamber into the tissue polyhedron suspension chamber, and wherein the method comprises removing tissue polyhedron suspension from the procurement chamber into the tissue polyhedron suspension chamber during or subsequent to step (b).

In one embodiment, step (b) is performed using the drive unit of the invention.

In one embodiment, the physiologically acceptable solution of the method of the present invention is selected from the group consisting of phosphate buffered saline (PBS), a physiological solution of sodium and potassium, a physiological solution of saline, a physiological solution of sodium, potassium magnesium and calcium, Ringer's solution, Ringer's lactate solution, physiological Hartmann solution, and a physiological solution comprising HEPES buffer.

In one embodiment, the method of the present invention may comprise the additional steps of centrifuging the suspension resulting from step (b) in the presence of density-gradient centrifugation components such as Percoll® solution to produce a gradient sediment of tissue fragments such as polyhedron-shaped tissue micro-transplants a supernatants; of removing the supernatant from the sediment of tissue fragments; and of suspending a selected gradient fraction in a physiologically acceptable solution, thereby producing a suspension comprising tissue fragments such as polyhedron-shaped tissue micro-transplants of selected density.

In one embodiment, the tissue fragments such as polyhedron-shaped tissue micro-transplants obtained from the donor tissue explant according to the invention are polyhedron-shaped. Preferably, the polyhedrons comprise viable cells of each cell type present in the donor tissue. For example, the polyhedrons comprise stem cells, supporting cells and biomatrix. In one embodiment, the polyhedron-shaped tissue micro-transplants of the tissue polyhedron suspension of the invention comprise adult stem cells, stem cell supporting cells, organ-typical parenchymal cells, supporting biomatrix and cell supporting factors/mediators/cytokines/hormones. Preferably, the stem cells, supporting cells and biomatrix in the polyhedrons are organized in a way that reflects their organization in the donor tissue. Thus, the viable cells of the polyhedrons comprise stem cells within an intact stem cell niche. This results in cells being able to outgrow from the polyhedrons and to differentiate into each cell type of the organ or tissue to be treated.

In one embodiment, the donor tissue used in the method of the present invention is or comprises roots of hair, mesenchymal tissue (including dermis and fat, skin tissue, cartilage, bone, muscle, heart muscle), epithelial tissue (including epidermal skin tissue), endothelial tissue (including vessels), ectodermal tissue, mesodermal tissue, endodermal tissue, and, optionally, bio-matrix components including proteins and proteoglycans, hormones, cytokines, mediators or growth factors. Thus, in one embodiment, the tissue polyhedron suspension of the invention comprises roots of hair, mesenchymal tissue (including dermis and fat, skin tissue, cartilage, bone, muscle, heart muscle), epithelial tissue (including epidermal skin tissue), endothelial tissue (including vessels), ectodermal tissue, mesodermal tissue, endodermal tissue, or roots and hair and, optionally, bio-matrix components including proteins and proteoglycans, hormones, cytokines, mediators or growth factors.

In one embodiment, the tissue polyhedron suspension is prepared by operating the device under conditions that result in processing and/or cutting the tissue into pieces of less than 3 mm, preferably less than 1 mm.

In one embodiment, the tissue polyhedron suspension that is prepared by the method of the present invention is sprayable and/or drippable.

In a seventh aspect, the present invention relates to a tissue polyhedron suspension according to the present invention, which is obtained or obtainable from the method described herein.

In an eight aspect, the present invention relates to a method of treatment of a patient comprising the step of administering the tissue polyhedron suspension of the present invention to the patient. The present invention also relates to the use of the tissue polyhedron suspension of the present invention for the preparation of a medicine or pharmaceutical composition for the treatment of a patient, preferably in a method comprising a step of administering the tissue polyhedron suspension, or a medicine or pharmaceutical composition comprising said tissue polyhedron suspension, to the patient.

In this eight aspect, the present invention also refers to the use of the tissue polyhedron suspension of the present invention for treating a patient, preferably by a method comprising a step of administering the tissue polyhedron suspension, or a medicine or pharmaceutical composition comprising said tissue polyhedron suspension, to the patient.

In one embodiment of the eight aspect, the method comprises a step of obtaining from the patient a biological sample comprising donor tissue and a further step of preparing the tissue polyhedron suspension from said donor tissue of the patient.

In one embodiment, the method comprises a step of obtaining from a donor a biological sample comprising donor tissue and a further step of preparing the tissue polyhedron suspension from the donor tissue of the donor, wherein said donor is not the patient.

In one embodiment, the tissue or the donor tissue is or comprises roots of hair, mesenchymal tissue including dermis and fat, cartilage, skin tissue, bone, muscle, heart muscle, epithelial tissue, endothelial tissue, ectodermal tissue, mesodermal tissue, or endodermal tissue, and, optionally, bio-matrix components including proteins and proteoglycans, hormones, cytokines, mediators or growth factors.

In one embodiment, the tissue polyhedron suspension of the present invention is sprayable and is administered by a spray-deposition device. Preferably, the tissue is or comprises skin, dermis of the skin or keratinocytes of the skin.

In one embodiment, the patient is affected from a skin wound, preferably an acute skin wound or a chronic skin wound, or an ulcer, and the method comprises spraying the tissue polyhedron suspension onto the wound or ulcer.

In one embodiment, the skin wound is a burn caused by fire, a friction burn, a cold burn caused by skin freezing, a thermal burn, a radiation burn, a chemical burn or an electrical burn.

In one embodiment, the wound of the patient receives an escharotomy prior to the spraying.

In one embodiment, the tissue polyhedron suspension of the present invention is administered by injection. Preferably, the injected suspension is or comprises roots of hair, mesenchymal tissue including dermis and fat, cartilage, bone, muscle, heart muscle, epithelial tissue, endothelial tissue, ectodermal tissue, mesodermal tissue, or endodermal tissue, and, optionally, bio-matrix components including proteins and proteoglycans, hormones, cytokines, mediators or growth factors.

In one embodiment, the method of treatment of the present invention comprises a further step of covering the treated wound with polylactid sheets, fatty gauze cotton, gauze, and/or elastic bandage. In one embodiment, the method of treatment of the present invention comprises a further step of covering the treated wound with sheets such as oxygen permeable silicon synthetic rubber sheets, membranes such as polylactic membranes, gauze such as fatty cotton gauze, liquid removing bandages, negative pressure generating and/or elastic bandage.

In another aspect, the invention relates to a kit comprising the device of the invention, the drive unit of the invention, a blade assembly and disposables. In one embodiment, the kit further comprises instructions for using the device and the drive unit for performing the method of the invention.

### BRIEF DESCRIPTION OF THE DRAWINGS

FIG. 1: “Tissue Procurement I”

A biological tissue micro-cutting device for tissue structure isolation to yield polyhedron-shaped micro-transplants according to the invention is shown. A rotating assembly of surgical blades connected to a motor is assembled in a procurement chamber for liquid immersion of tissue. (a)-(c) show three of many variations of the chamber, or liquid and tissue container. (d) shows one embodiment of a blade assembly, using surgical of-the-market disposable blades.

FIG. 2: “Tissue Procurement II”

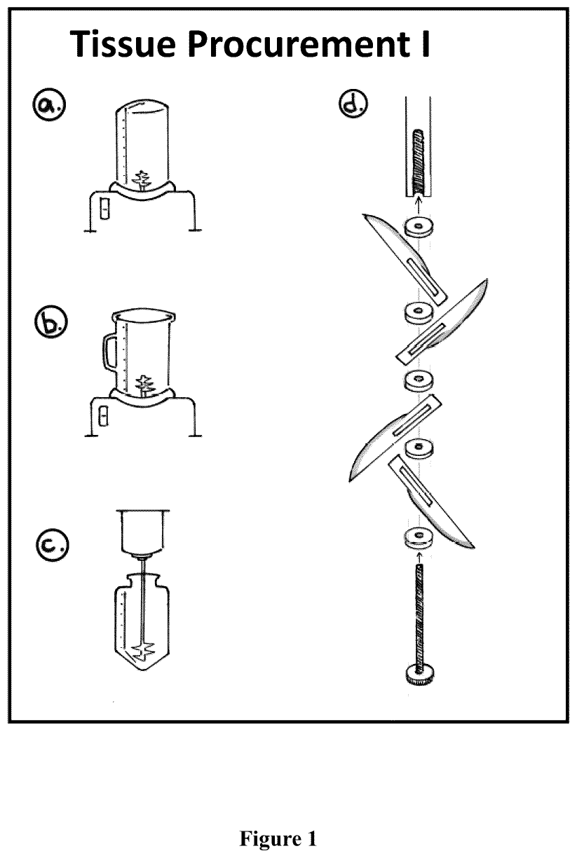

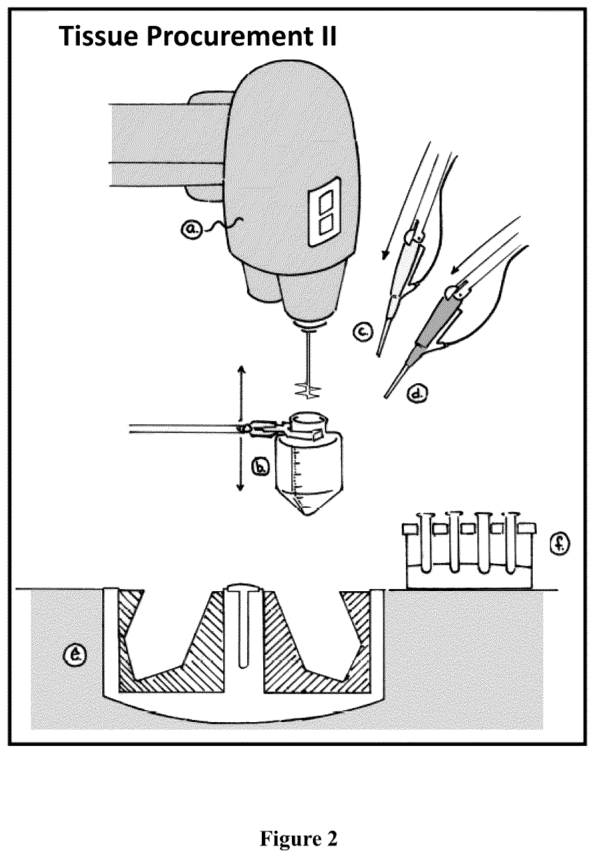

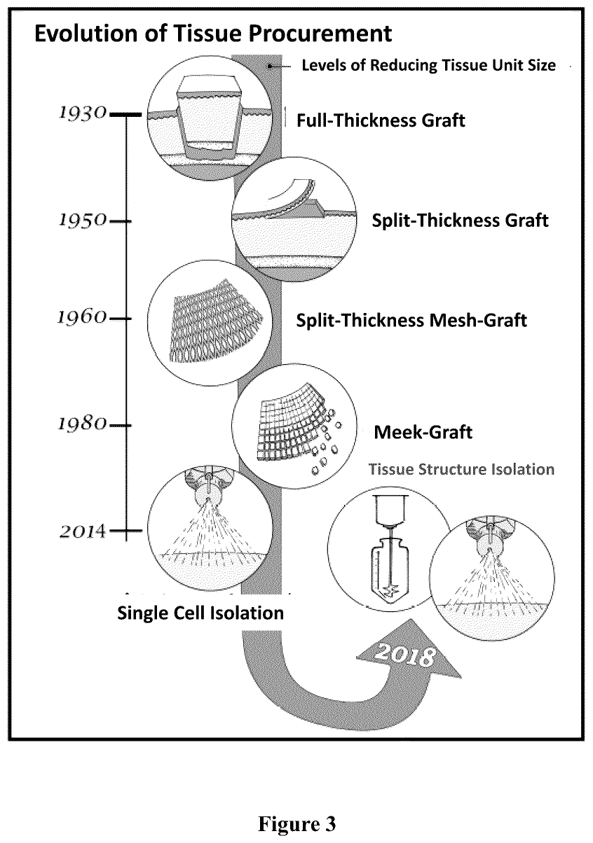

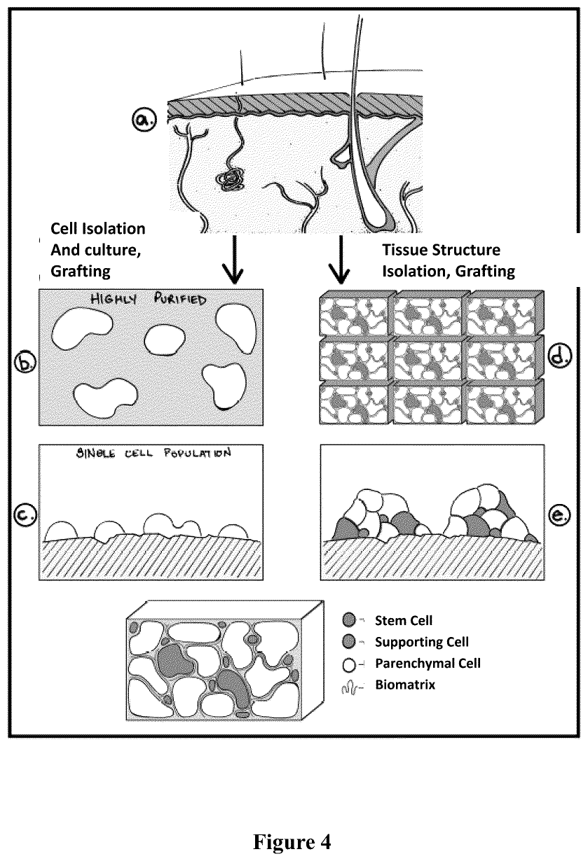

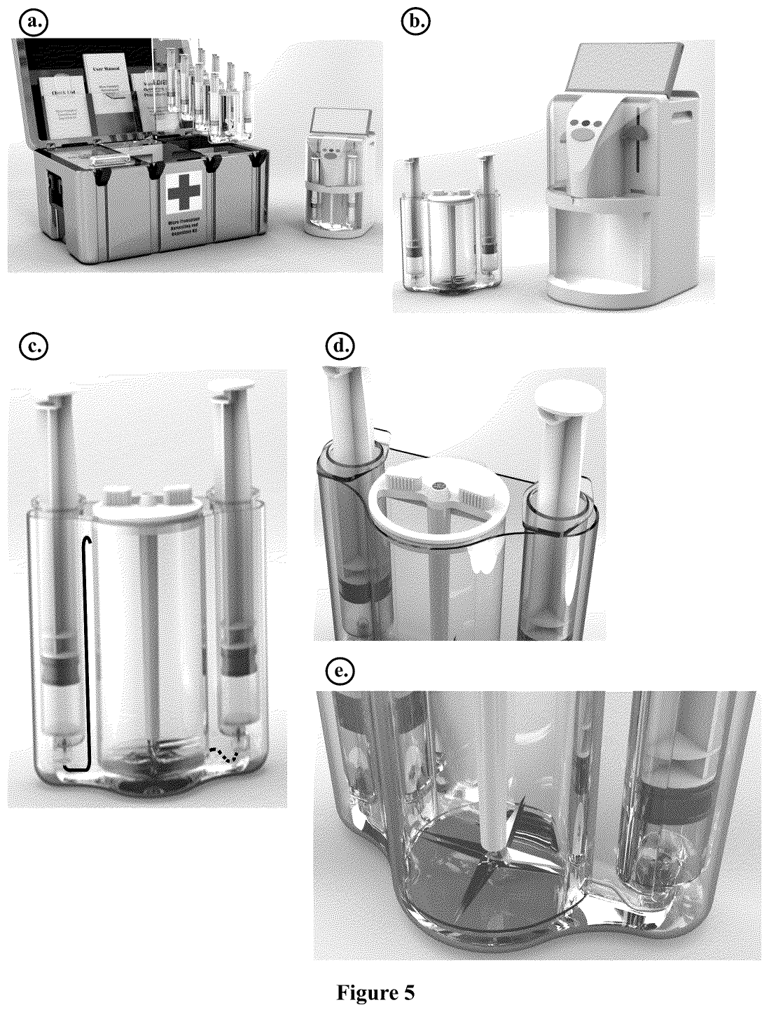

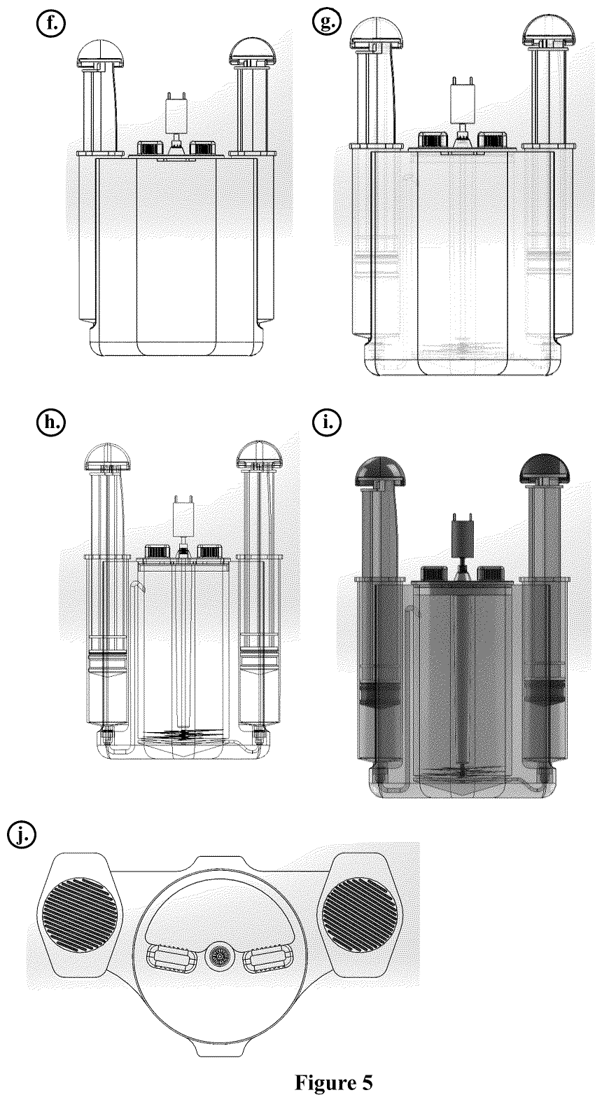

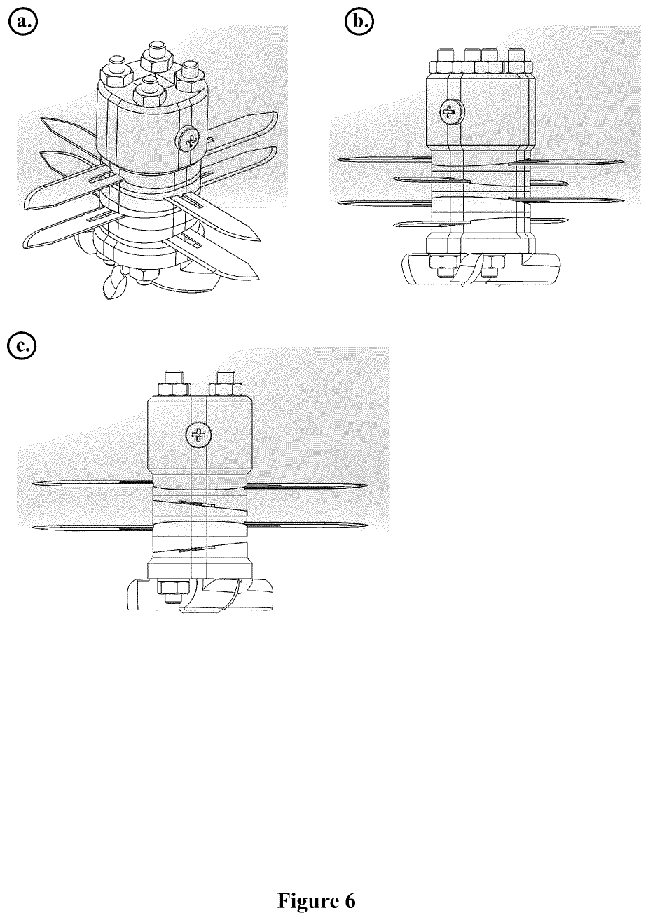

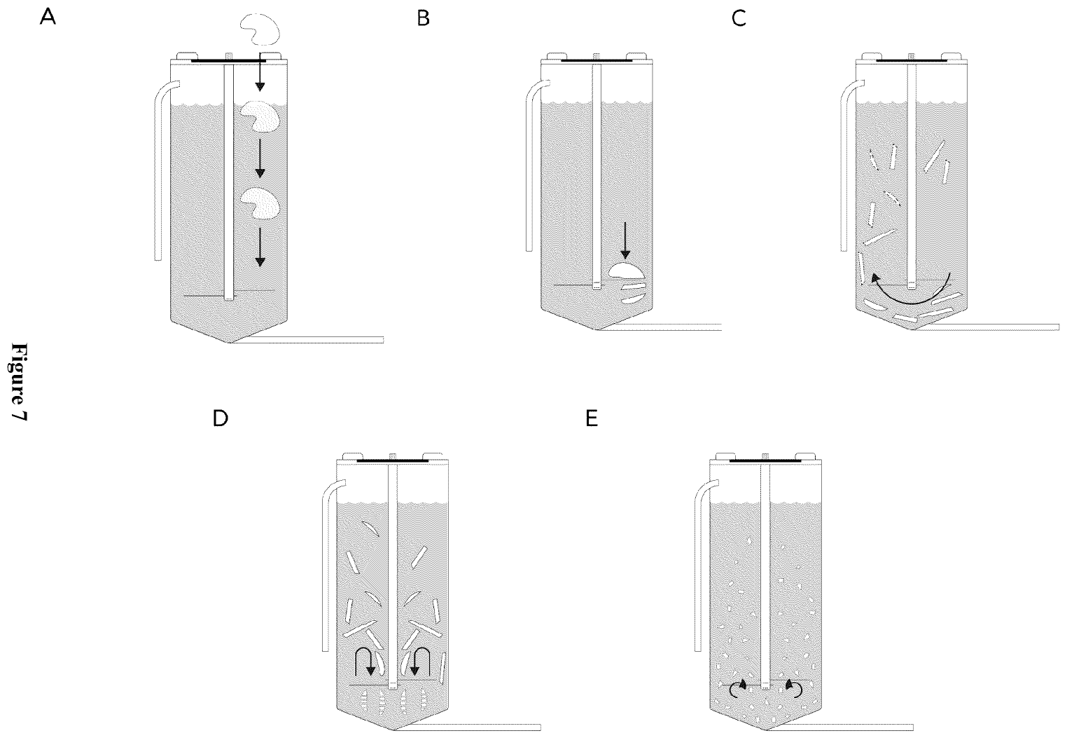

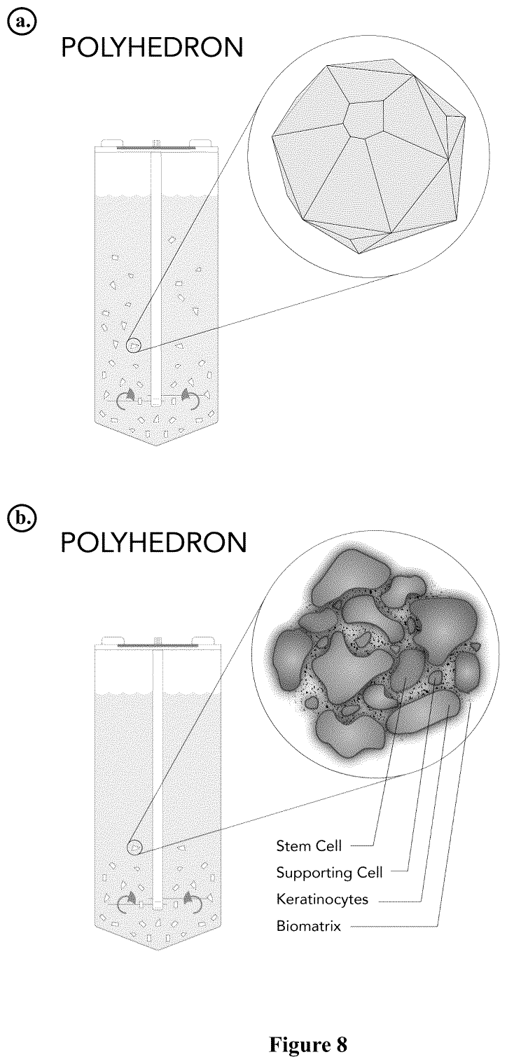

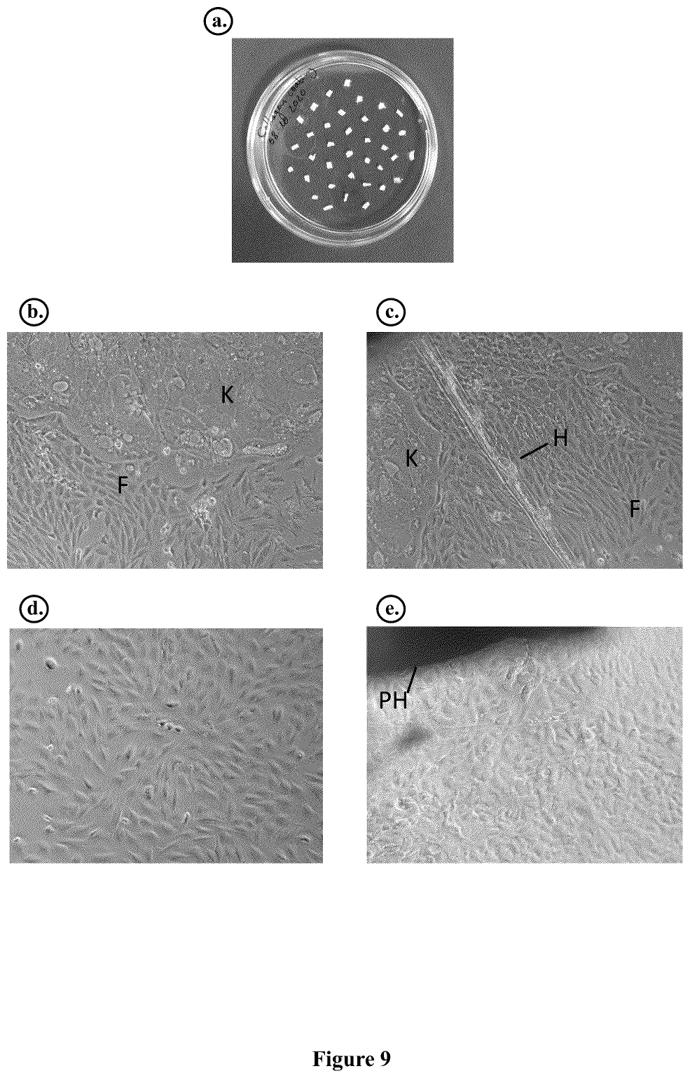

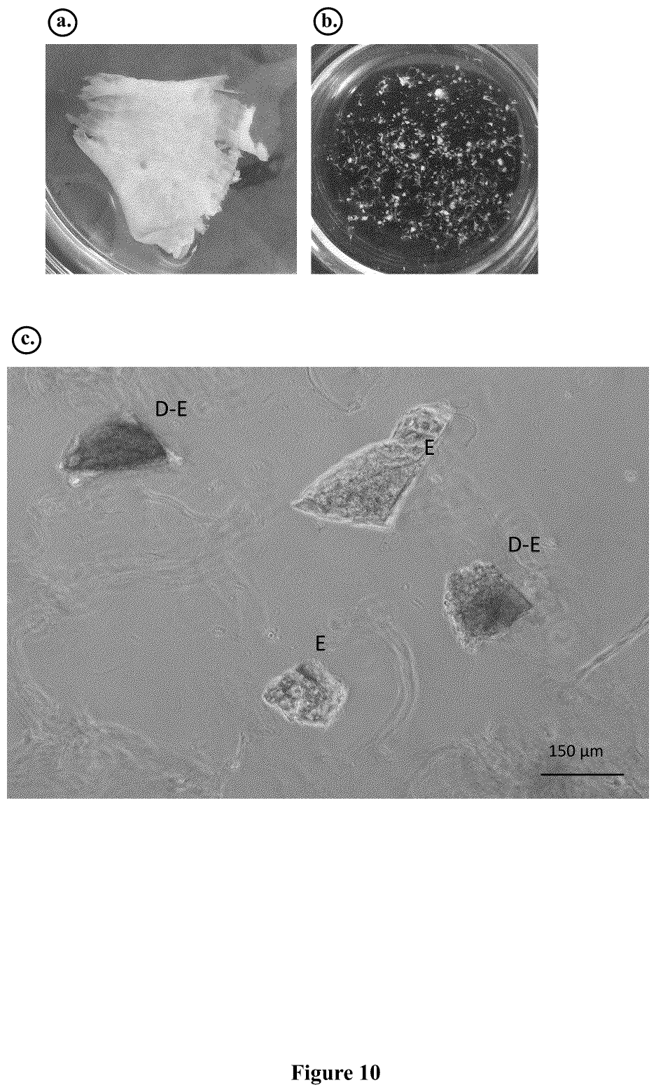

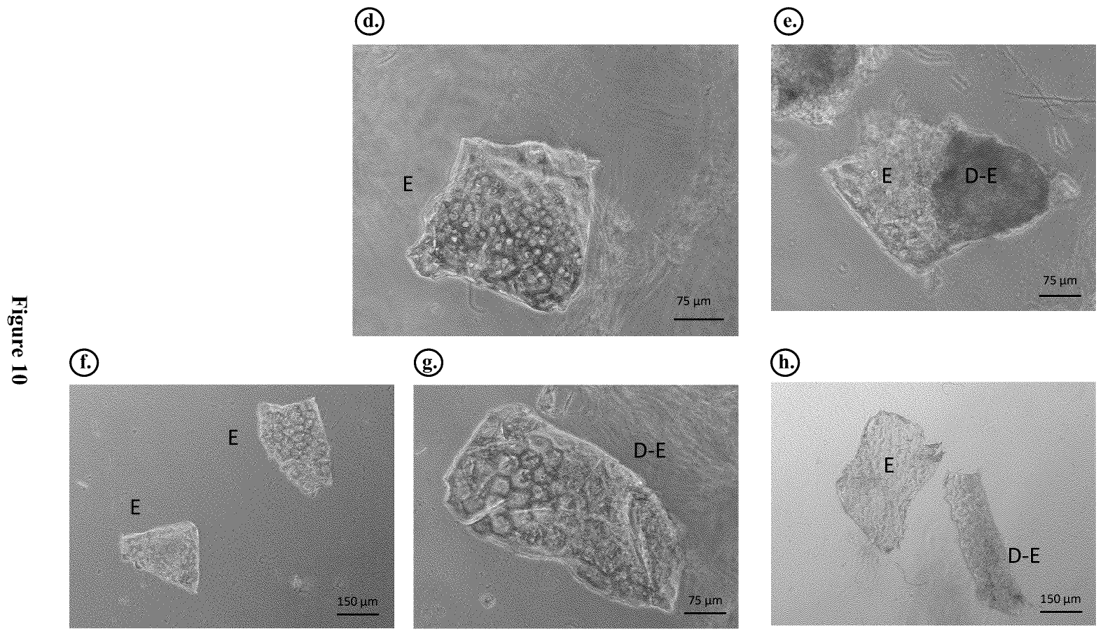

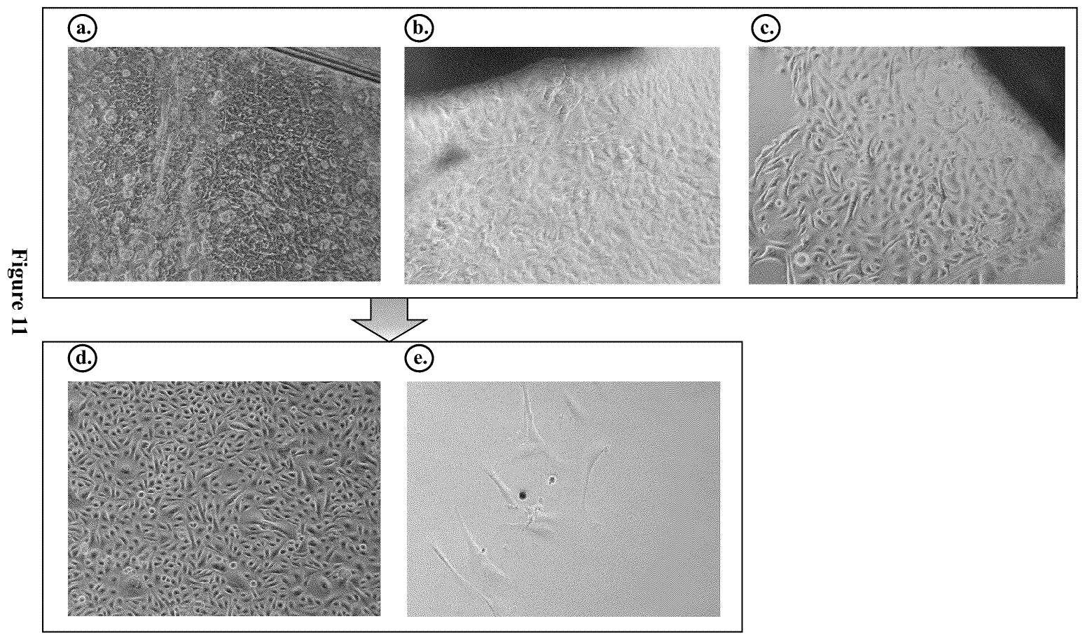

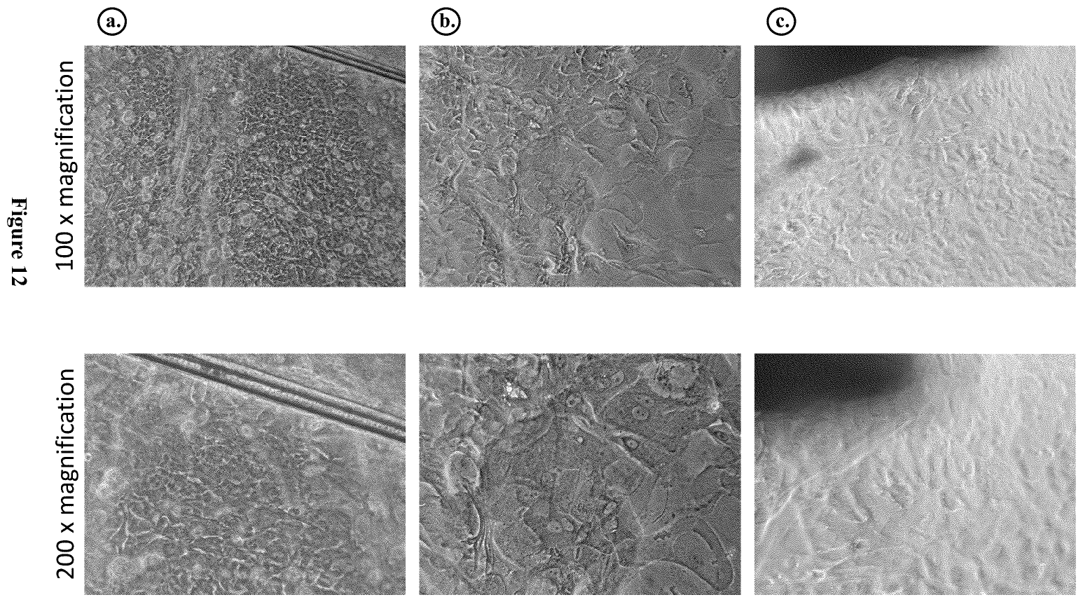

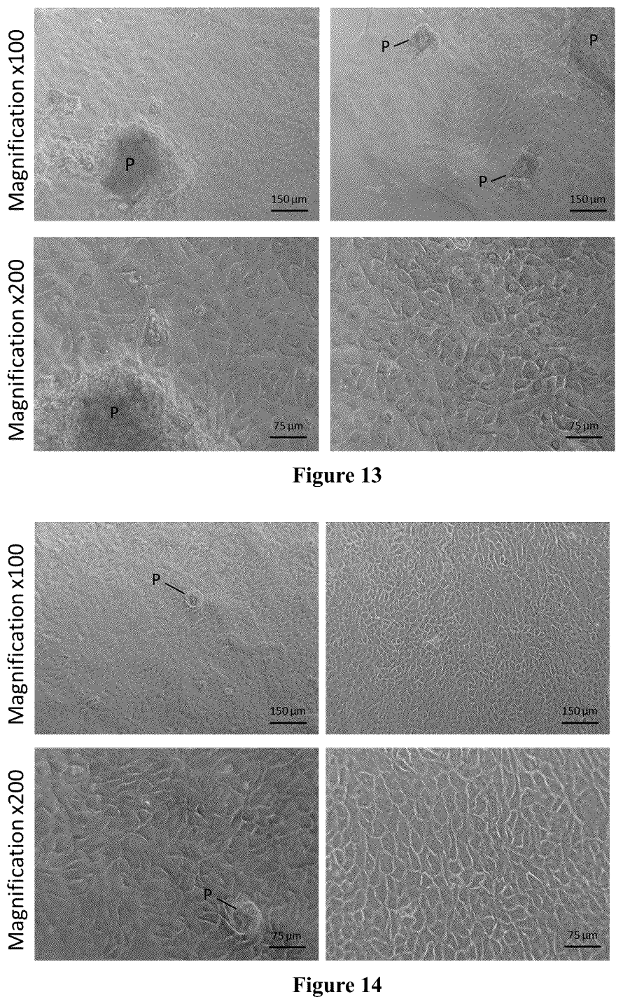

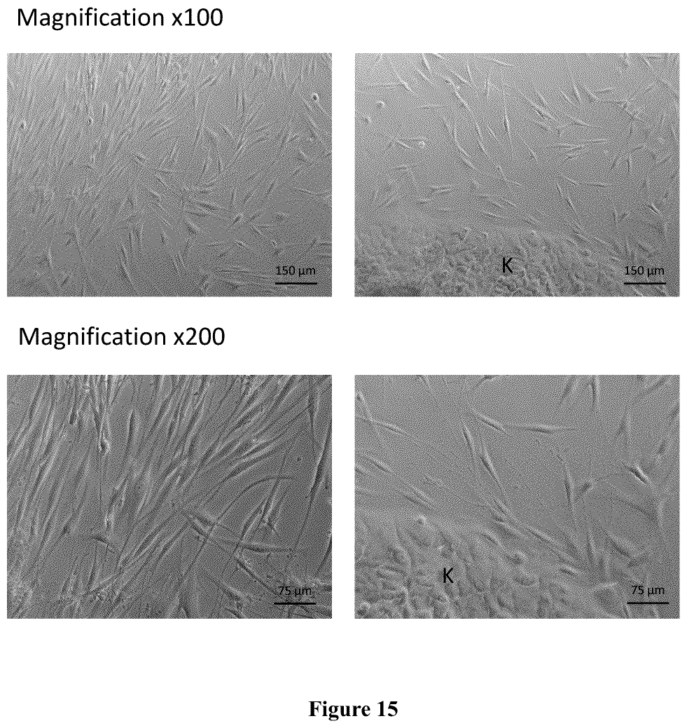

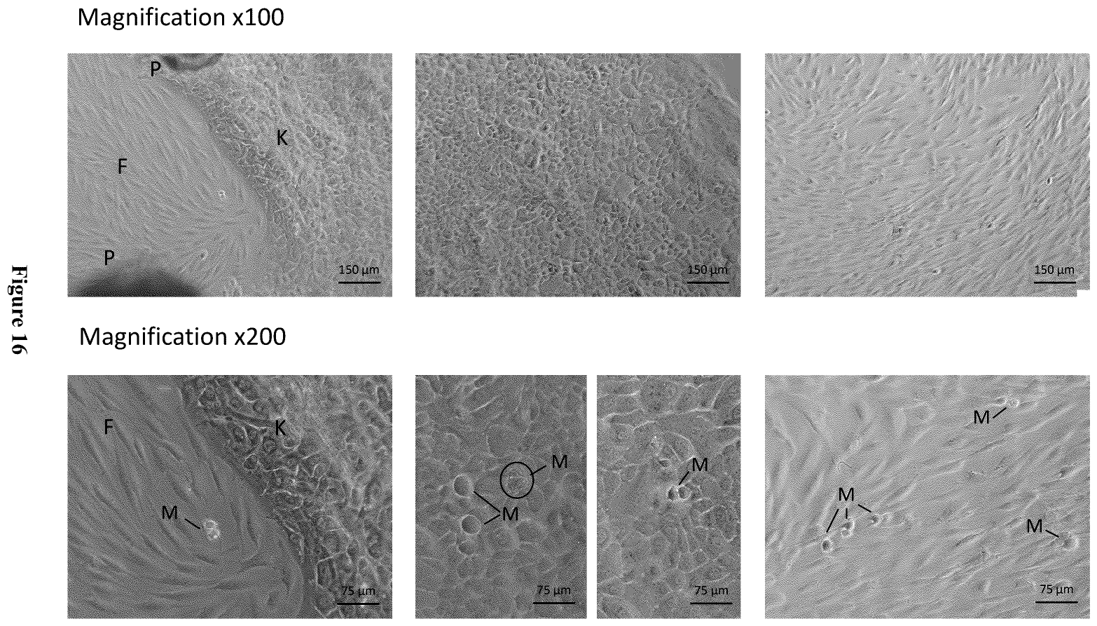

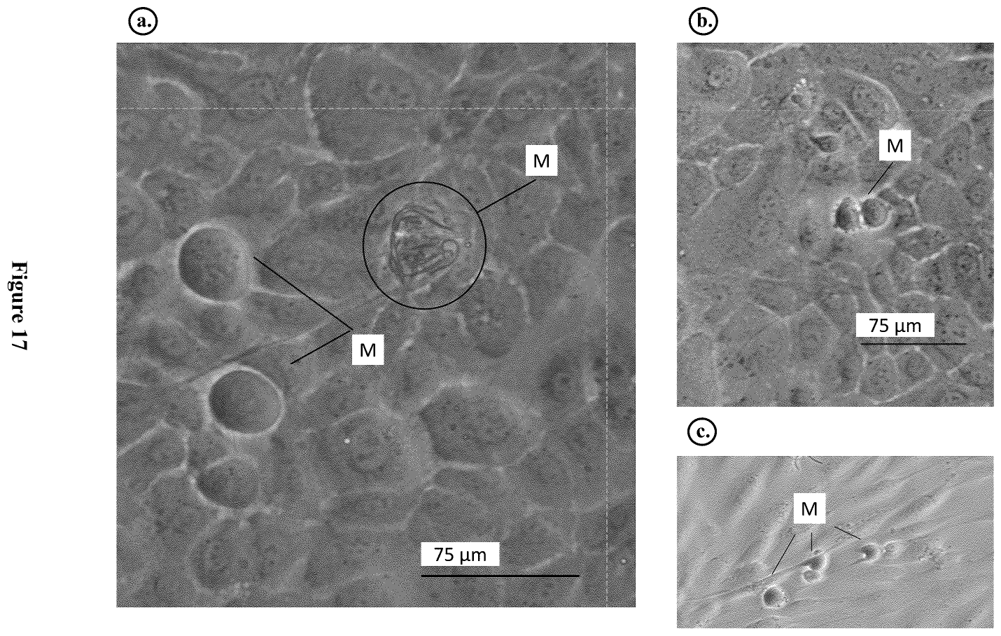

A motor (a) that is connected with blades as shown in FIG. 1 is mounted statically and a procurement chamber (b) that is described in FIG. 1 is mounted on a movable bracket that facilitates automation. The chamber, or procurement chamber, for liquid immersion of tissue can be filled with, e.g. saline solution and the tissue to be cut can be added. The procurement chamber can move up and down for immersion of the blades to enable cutting. The movable bracket can feature an automated grip and can be moved up and down to insert the blades from the top. Automated pipettes (c, d) can add or remove liquid or the resulting tissue micro cube suspension. The movable bracket (b) can feature an automated grip and thus can be moved up and down into a centrifuge (e) and position the procurement chamber there, or remove it again. Automated pipettes (c, d) can remove supernatant liquid or the resulting tissue micro cube suspension. These automated pipettes can dispose the resulting micro cubes in a further procurement chamber (f), e.g. for tissue micro cube deposition to the target tissue.

FIG. 3: “Evolution of Tissue Procurement”

A timeline is given and inventions at each time period are illustrated. The novel technique “tissue structure isolation using a micro-cutting device” and an exemplary use of resulting micro-cubes of tissue for spray deposition is shown at the bottom right. The time arrow is bend to the right and upward, as the novel technique represents a step back from the isolation of single cells to providing tissue fragments, as is today mostly used with the “split thickness mesh graft” technique depicted, that would not allow for spray deposition.

FIG. 4: “Comparison of Tissue Procurement between enzymatic single cell isolation, the state of the art—with the novel tissue structure isolation using a micro-cutting device”

(a) shows the tissue to be manipulated, (b) and (c) indicate steps of the enzymatic method, (d) and (e) indicate steps of the novel method. (b) shows resulting single cells resulting from the enzymatic method, floating in a suspension, where only one cell type was isolated and the biomatrix is lost. (d) shows micro-cut cube-like tissues that contain all cells of the original tissue together with their surrounding biomatrix floating in a suspension. (c) shows how single cells adhere to tissue, immobilize and spread to form a monolayer. (e) shows how the microcubes adhere to tissue immobilize and form a cluster of composite tissue, more resembling the original situation in the original tissue.

FIG. 5: “Tissue Procurement Device, Drive Unit and Kit comprising the same”

(a) shows a kit comprising a device for preparing a tissue polyhedron suspension from a biological sample comprising donor tissue according to the invention and a drive unit configured to accept the device. In the depicted embodiment, the device comprises a liquid chamber configured to receive liquid and to provide the liquid into the procurement chamber, and a tissue polyhedron suspension chamber configured to accept tissue polyhedron suspension, and optionally to remove the tissue polyhedron suspension from the procurement chamber into the tissue polyhedron suspension chamber, wherein both the liquid chamber and the tissue polyhedron suspension chamber are depicted in the form of syringes. The drive unit comprises buttons allowing to control parameters of the drive unit and a screen for providing information to the user. (b) shows the device and drive unit shown in (a) side by side. (c) shows the fluid communication way between the liquid chamber (left side, solid line) and between the tissue polyhedron suspension chamber (right side, dotted line) and the procurement chamber (middle). (d) is a view onto the device showing the first opening which is in the depicted embodiment equipped with a means for closing the first opening in the form of a closing lid comprising a rotatably mounted portion as described herein, wherein the means for closing the first opening in the depicted position allows addition of material such as donor tissue through the feed-through into the procurement chamber of the device and in another position (not shown) acquired by rotating the upper portion of the closing means closes the first opening such that no addition of any material is possible. The closing lid comprises a bore such as a central bore and the polyhedron generating tool is arranged in the closing lid in such a way that it is rotatably mounted through a bore such as the central bore of the closing lid. (e) depicts the blade assembly of one embodiment, wherein the polyhedron generating tool comprises four micro-surgery scalpel blades. Of course, the number of blades is not particularly limited as long as the blade assembly allows obtaining polyhedron-shaped tissue fragments comprising viable cells of all cell types present in the donor tissue used. (f) is a line drawing depicting the shape of the device of the present invention. (g-i) are line drawings depicting the shape of the device of the present invention, wherein the liquid chamber (left), tissue polyhedron suspension chamber (right) (both chambers depicted as syringes), procurement chamber with lid, polyhedron generating tool within the procurement chamber, agitating element (protruding through the lid) and means for engagement of the agitating element. j) is a top view of one embodiment of the device of the invention showing the lid and the feed-through. This assembly can be offered as single-use sterile disposable.

FIG. 6: “Polyhedron generating tool comprising a holding element”

Depicted is a polyhedron generating tool comprising a holding element. The holding element comprises a body comprising a stack of segments. The blades are held between segments of the stack of segments by press fit. (a) the blades are arranged in two groups of blades wherein the blades of each group have a cross-like arrangement. (b) Several segments of the stack of segments have undulated upper and/or lower faces. (c) a blade of the upper group of blades is arranged such that its cutting edge is higher that its blunt edge while the respective blade from the lower group of blades is arranged such that its cutting edge is lower that its blunt edge. This element can be offered as part of a single-use sterile disposable.

FIG. 7: “Processing of Donor Tissue”

The process of processing donor tissue into a tissue polyhedron suspension in polyhedron form using the device of the invention is depicted. (a) First, the donor tissue is added into the procurement chamber and is pulled down by gravity. (b) The device generates turbulences and subsequently, the donor tissue is processed into smaller fragments. (c-e) By gravity and by the turbulences generated through the device, the tissue fragments re-enter the process of tissue processing (polyhedron generating) multiple times and are processed into ever smaller fragments, ultimately resulting in tissue fragments in polyhedron form which show a cellular organization as shown in FIG. 6.

FIG. 8: “Polyhedron Morphology”

(a) shows a geometrically idealized depiction of a typical tissue fragment in the form of a polyhedron obtained by processing donor tissue using the device of the invention. (b) shows the cellular composition of a typical tissue fragment in the form of a polyhedron obtained by processing donor tissue using the device of the invention.

FIG. 9: “In vitro culture of porcine skin polyhedrons, phase contrast microscopy”

In a first in vitro study, pig skin derived polyhedrons were generated to establish an in vitro model. This was followed by an in vitro study observing the behavior of resulting human skin cells in culture (FIG. 10). The micro-transplants were generated as very large polyhedrons and taken into in vitro culture Petri-dishes, to observe their culture behavior as an in vitro model of skin wound healing. The culture was performed in uncoated culture dishes using DMEM high glucose+10% FCS culture medium (DMEM) typically used for mesenchymal fibroblasts. Porcine keratinocyte culture was enabled best using DMEM medium, human keratinocyte medium with 10% FCS (KCM) did only support human keratinocytes. (a) Very large polyhedrons (2×2 mm²) from porcine skin. (b-e) magnification×100. (b) From skin, at least two populations can be described: epidermal keratinocytes and mesenchymal fibroblasts, both were able to be transferred and the grew in microscopically (phase-contrast) visible clusters, as expected. Mixed fibroblasts (F) and keratinocytes (K) outgrowth from polyhedron, day 9 after start of in vitro culture. (c) Hair could be transferred. Fibroblasts (F), hair (H), keratinocytes (K), KCM, day 9 after start of in vitro culture. Keratinocytes (from the so-called epithelial layer of the skin) migrate out of the polyhedrons and grow in Petri-dishes. The cells reoriented themselves actively in the 3D space, after migrating out of the polyhedron, into a layer structure resembling the epithelial layer of the skin. They survived several weeks and could be passaged from dish to dish. (d) Fibroblasts, DMEM, day 18, typical porcine morphology. (e) The cells migrate out of the polyhedrons and grow to form a monolayer on the culture surface. Keratinocytes in DMEM, day 17 after start of in vitro culture, Polyhedron is marked (PH). Mesenchymal cells (that live under the epidermal layer) could be transferred as well. Cells were able to actively leave the polyhedron, migrate over the dish surface and then undergo mitoses to grow in number, towards forming a so called monolayer on the dish surface. This was a first in vitro proof of principle, of keratinocytes outgrowing from a polyhedron produced by the method of the invention using the device of the invention and repairing a wound by growing from the healthy edge of the wound towards the center of the wound and form there a layer of new keratinocytes, the so called re-epithelialization. Devices of the prior art are not able to produce tissue polyhedron suspension harboring such capabilities.

FIG. 10: “Culture of human skin polyhedrons—in vitro survival over two weeks and morphology”

In a second in vitro study, human skin derived tissue was used and polyhedrons generated in vitro. This was followed by an in vitro study using human skin cells to observe their culture behavior. The micro-transplants were generated with the described device in comparison to FIG. 9 in a smaller size and taken into in vitro culture Petri-dishes, to observe their culture behavior as an in vitro model of skin wound healing.

Skin samples were immerged in 100 ml Ringer's Lactate solution in the device. Cutting blade spinning was performed at 20,000-30,000 rpm, for 20-40 sec. Polyhedrons were seeded into uncoated or collagen-coated culture flasks using keratinocyte culture medium with fetal calf serum (FCS) or DMEM high glucose with FCS. N=3 repeats with individual human skin samples (ca. 10 cm² tissue area). (a) Human skin sample (ca. 10 cm²) used for harvesting 3D tissue super-structures (polyhedrons). Phase-contrast microphotographs showing human skin tissue that was processed to a polyhedron. The square-like outer shape is a result of the specific procurement using the method and device of the invention. Multiple cell nuclei are visible. Some liberated connective tissue fibres float close to the polyhedron. (b) Polyhedrons harvested and seeded in culture dishes for in vitro culture. (c-h) 3D tissue super-structures after using the device for tissue procurement; polyhedrons seeded into culture flasks and 48 h in culture; phase-contrast microscopy, for magnification see bars; E=epidermal structures, showing typical keratinocyte patterns, D-E=dermal-epidermal structures, containing connective tissue (brown); phase-contrast microscopy showing human skin tissue that was processed to a polyhedron, before taking into culture. The angular outer shape representing polyhedrons is a result of the specific procurement using the principle and device of the invention. Multiple cell nuclei are visible. Some liberated connective tissue fibres float close to the polyhedron. Phase-contrast microscopy showing human skin tissue that was processed to a polyhedron, before taking into culture. The angular outer shape representing polyhedrons is a result of the specific procurement using the method and device of the invention. Multiple cell nuclei are visible. Some liberated connective tissue fibres float close to the polyhedron.

FIG. 11: “Culture of human skin polyhedrons”

In a third study, primary cultures of the resulting polyhedrons were studied versus the outgrown cells out of primary polyhedron cultures which were then passaged into subsequent cultures. This was initiated to confirm comparable cell morphology in primary culture and follow-up culture. Also, this was performed in order to demonstrate that the resulting polyhedrons behave in in vitro culture comparably to conventional cultures of such cells. Cultures were performed in uncoated culture dishes using DMEM high glucose+10% FCS (DMEM) or keratinocyte culture medium+10% FCS (KCM), magnification×100. (a-c): Primary culture, cell outgrowth onto a Petri-dish surface from polyhedrons. (a) Keratinocytes, DMEM & FCS, day 17. (b) Keratinocytes, DMEM, day 17. (c) Keratinocytes, KCM & FCS, day 16. (d-e) Passaged cells, from dish to second dish, follow-up. (d) Passage 1, keratinocyte cond., KCM & coated, no FCS, day 18. (e) Passage 1, fibroblast conditions, day 21, low density culture, fibroblast medium DMEM & FCS, uncoated. The results show that the cells actively leave the polyhedrons and are able to “repair” an empty dish surface by actively migrating over it, forming a mono-layer as desired for wound healing by re-epithelialization. The cells showed that they can perform such complex tasks and, surprisingly, behaved as normally expected from conventional primary cultures.

FIG. 12: “Human polyhedrons obtained according to the invention comprise viable cells”

Human skin tissue samples were processed using the device of the invention. Human skin tissue polyhedrons at day 17 of culture are shown. The polyhedrons where cultured in uncoated flasks with DMEM, high glucose+10% FCS. (a) shows a monolayer of epithelial cells comprising islets of cells of other morphology outgrown from polyhedrons. Part of a hair can be seen in the upper right corner indicating that viable cells of all cell types present in the respective donor tissue were present in the polyhedron. (b) shows cell islets containing at least two different types of cells. (c) shows cells growing out from a polyhedron indicating that the cells are viable. Devices of the prior art such as those disclosed in WO 2012/083260, WO 2013/070899, WO 2016/097960, WO 2010/036788, US 2011/282242 and WO 2019/147414 are not capable to produce such results and their use does not result in generation of polyhedron-shaped micro transplants.

FIG. 13: “Follow-up in vitro culture of human skin polyhedrons”

3D tissue super-structures on day 5 of culture. Outgrowth and expansion of keratinocytes around polyhedrons (P).

FIG. 14: “3D tissue super-structures on day 9 of culture of human skin polyhedrons”

Formation of large confluent keratinocyte layers outgrowing from polyhedrons (P). Some remaining polyhedrons are embedded in the surrounding cell layer.

FIG. 15: “3D tissue super-structures on day 14 of culture of human skin polyhedrons”

Proliferation of fibroblasts around keratinocyte layers (K) is visible.

FIG. 16: “3D tissue super-structures on day 14-16 of culture of human skin polyhedrons”

Confluent keratinocyte (K) and fibroblast (F) layers showing active cell proliferation. M=mitoses in different stages of cell division; P=polyhedrons

FIG. 17: “Cells from human skin polyhedrons regenerate a monolayer when actively growing out of the 3D”

Tissue super-structures and actively proliferate in number for tissue repair. (a-c) Magnification×200, M=mitoses (regenerative cell divisions), day 16 of culture. (a) Epidermal cells prepare for mitosis; circle: mitotic chromatin with chromosomes in spindle-like array migrating to form the nuclei of the new two daughter cells. (b) daughter cells reintegrate into monolayer to complete mitosis; epidermal cells. (c) comparable processes can be observed with dermal cells.

### DETAILED DESCRIPTION

Although the present disclosure is described in detail below, it is to be understood that this disclosure is not limited to the particular methodologies, protocols and reagents described herein as these may vary. It is also to be understood that the terminology used herein is for the purpose of describing particular embodiments only, and is not intended to limit the scope of the present disclosure which will be limited only by the appended claims. Unless defined otherwise, all technical and scientific terms used herein have the same meanings as commonly understood by one of ordinary skill in the art.

In the following, the elements of the present disclosure will be described. These elements are listed with specific embodiments. However, it should be understood that they may be combined in any manner and in any number to create additional embodiments. The variously described examples and embodiments should not be construed to limit the present disclosure to only the explicitly described embodiments. This description should be understood to disclose and encompass embodiments which combine the explicitly described embodiments with any number of the disclosed elements. Furthermore, any permutations and combinations of all described elements should be considered disclosed by this description unless the context indicates otherwise.

The term “device of the invention” relates to the device of any and all aspects of the invention, e.g., to the device according to the second, third, fourth aspect of the invention. Thus, embodiments relating to the “device of the invention”, or to parts thereof, are combinable with the device of any and all aspects of the invention. As will be understood, the same holds true for further features of the invention. For example, the term “polyhedron-shaped tissue micro-transplants of the invention” relates to any and all aspects and embodiments relating to polyhedron-shaped tissue micro-transplants. As further examples, embodiments relating to the polyhedron generating tool, or to any chamber (including the procurement chamber, liquid chamber and tissue polyhedron suspension chamber), or to any means are combinable with the device according to any and all aspects of the invention and any and all embodiments described herein. The embodiments and aspects described herein are combinable unless explicitly stated otherwise or clearly technically incompatible.

The term “about” as used herein means approximately or nearly, and in the context of a numerical value or range set forth herein in one embodiment means ±20%, ±10%, ±5%, or ±3% of the numerical value or range recited or claimed.

The terms “a” and “an” and “the” and similar reference used in the context of describing the disclosure (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it was individually recited herein. All methods described herein can be performed in any suitable order unless otherwise indicated herein or otherwise clearly contradicted by context. The use of any and all examples, or exemplary language (e.g., “such as”), provided herein is intended merely to better illustrate the disclosure and does not pose a limitation on the scope of the claims. No language in the specification should be construed as indicating any non-claimed element essential to the practice of the disclosure.

Unless expressly specified otherwise, the term “comprising” is used in the context of the present document to indicate that further members may optionally be present in addition to the members of the list introduced by “comprising”. It is, however, contemplated as a specific embodiment of the present disclosure that the term “comprising” encompasses the possibility of no further members being present, i.e., for the purpose of this embodiment “comprising” is to be understood as having the meaning of “consisting of”.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the present disclosure was not entitled to antedate such disclosure.

In the following, definitions will be provided which apply to all aspects of the present disclosure. The following terms have the following meanings unless otherwise indicated. Undefined terms have their art-recognized meanings.

As used herein, the term “tissue polyhedron suspension”, refers in particular to an aqueous solution, preferably a physiologically acceptable solution, comprising fragments of tissue. The term “tissue polyhedron suspension” is used to distinguish the suspension of the present invention from a “suspension of single cells”: while the former essentially comprises tissue fragments such as polyhedron-shaped tissue micro-transplants composed of interacting cells embedded in their natural biomatrix, the latter essentially comprises single cells. The term “tissue fragment” as used herein, preferably refers to a product obtainable by fragmentation or processing (e.g., by cutting) of tissue. The term includes tissue fragments of any kind of regular or irregular form or shape. The tissue fragments of the suspension of the present invention exhibit an outer shape or form that results from a multitude of cuts, i.e., a polyhedron shape. Preferably, the fragments are irregular, i.e. polyhedron-shaped and can comprise a multitude of flat cut surfaces, together with more spherically cut surfaces.

The fragments in the tissue polyhedron suspension of the present invention such as the polyhedron-shaped tissue micro-transplants prepared by using the device of the present invention are polyhedrons comprising cells, preferably viable cells, of each cell type present in the procured tissue. Thus, in one embodiment, the polyhedron-shaped tissue micro-transplants of the invention, e.g, as obtained by the method of the invention, comprise adult stem cells, stem cell supporting cells, organ-typical parenchymal cells, cell supporting biomatrix and cell supporting factors/mediators/cytokines/hormones. A typical polyhedron of the tissue polyhedron suspension of the invention is depicted schematically in FIG. 8. In one embodiment, the tissue fragments of the tissue polyhedron suspension of the present invention have been obtained by using the device of the present invention. In one embodiment, the polyhedrons comprise stem cells, supporting cells and surrounding biomatrix, wherein the cells and biomatrix are organized in a way that reflects their organization in the donor tissue as naturally occurring, e.g., in a host organ, the respective polyhedrons are derived from. In one embodiment, the polyhedrons comprise stem cells, supporting cells, keratinocytes and biomatrix. In one embodiment, the organization of the stem cells, supporting cells, keratinocytes and biomatrix is similar to the organization of the stem cells, supporting cells, keratinocytes and biomatrix as found in the tissue the polyhedrons are derived from. The polyhedrons of the tissue polyhedron suspension have a size that allows spraying the tissue polyhedron suspension using a spray device. Any spray device for spraying a tissue polyhedron suspension known to the skilled person can be used.

The polyhedrons of the tissue polyhedron suspension of the present invention comprise a substantial number of viable cells. Preferably, the natural cell population distribution of the viable cells in the polyhedrons of the tissue polyhedron suspension is preserved.

The term “at least 100” as used throughout the present teaching encompasses for example at least 150, at least 200, at least 300, at least 400, at least 500 or at least 1000. Also encompassed are any kind of ranges with a lower limit of for example 100, 200, 300, 400, 500, 600, 700, 800, 900 or 1000 or an upper limit of up to 200, 300, 400, 500, 600, 700, 800, 900, 1000, 2000, 3000, 4000, or 5000. Specific examples include ranges such as 100-200, 100-500, 100-1000, 500-1000, 500-2000, 500-5000 and so on.

In a preferred embodiment, the “tissue fragment” of the present invention includes “polyhedron” and “polyhedron-shaped tissue micro-transplant” and comprises an average of at least 50 cells, at least 75 cells, at least 100 cells, at least 150 cells, at least 200 cells, at least 300 cells, at least 400 cells, or at least 500 cells. However, for some embodiments it is preferred to limit the cell number in the tissue fragment such as polyhedron-shaped tissue micro-transplant to an average of up to 100 cells, up to 200 cells, up to 300 cells, up to 400 cells, up to 500 cells or up to 1000 cells. An “average of up to 100 cells”, means that a suspension may comprise for example a mixture of tissue fragments such as polyhedron-shaped tissue micro-transplants of sizes such as 80cells, 90 cells, 100 cells, 110 cells and 120 cells which amounts to a calculated average of 100 cells per average tissue fragment. An “average of up to 1000 cells”, means that a suspension may comprise for example a mixture of tissue fragments such as polyhedron-shaped tissue micro-transplants of sizes such as 800 cells, 900 cells, 1000 cells, 1100 cells and 1200 cells which amounts to a calculated average of 1000 cells per average tissue fragment. Typically, the average includes a variation from a mean or average size by up to 5%, up to 10%, up to 15% or up to 20%. Preferably, a tissue fragment is a polyhedron such as a polyhedron-shaped tissue micro-transplant.

In another preferred embodiment, the tissue polyhedron suspension comprises a mixture of sizes of tissue fragments such as polyhedron-shaped tissue micro-transplants. This mixture preferably falls into an interval of between about 50-1000 cells, or of between about 100-500 cells, or of between about 250-500 cells.

Any of the aforementioned cell numbers can be determined in conventional assays such as microscopy based on tissue staining with hematoxylin and eosin stain (H&E) and volume extrapolation, or trypsinization and single cell counting using a hemocytometer. These assays are typical reference assays for determining the aforementioned cell numbers.

The term “tissue fragment”, “polyhedron” and “polyhedron-shaped tissue micro-transplant” as used herein particularly refers to a tissue fragment such as a polyhedron-shaped tissue micro-transplant comprising a cellular component and, optionally, a non-cellular component, wherein a tissue fragment such as a polyhedron-shaped tissue micro-transplant of the suspension of the present invention is characterized by essentially preserving the composition and/or many or all of the intercellular interactions of the host tissue from which they are derived. Preferably, the cell types and/or the non-cellular components in the suspension of the present invention are identical to donor tissue from which the tissue fragments such as polyhedron-shaped tissue micro-transplants were generated. Typically, the tissue fragments such as a polyhedron-shaped tissue micro-transplant of the suspension of the present invention comprise macromolecular structures such as tight junctions, gap junctions, desmosomes, macula adherens and/or other structures typically observed in the donor tissue from which the tissue fragments such as polyhedron-shaped tissue micro-transplants are derived. For example, a tissue fragment such as polyhedron-shaped tissue micro-transplant obtained from the skin would comprise the cells of the stratum corneum, the cells of the granular layer, the cells of the spinous layer, the cells of the basal layer, the cells of the dermis. Thus, the suspension of the present invention when obtained from skin donor tissue can comprise various cell types. These cellular components of the suspension of the present invention include for example keratinocytes, melanocytes, melanocyte stem cells, Langerhans cells, Merkel cells, cells forming the arrector pili muscle, cells of sensory nerves, T cells, mast cells, dendritic cells of the skin, dermal fibroblasts, adipocytes, dendritic epidermal T cells, cells forming dermal blood vessels. In addition, however, the suspension of the present invention when prepared from skin tissue may comprise a number of non-cellular components of the skin such as EGF, FGF-7, FGF-10, IGF-1, TGFα, EGFR ligands, Notch1, Notch2, Notch3, Laminin-5, integrins such as α₃β₁ and α₆β₄, involucrin, keratins, cadherins, lipids, collagens, fibronectins, and the like.

The present teaching excludes in a preferred embodiment suspensions of tissue fragments comprising a substantial amount of single cells. The term “substantial amount of single cells” preferably refers to an amount of more than 15% of single cells in comparison to the overall cell number. In another preferred embodiment, the present invention also excludes suspensions comprising tissue fragments which measure in any one of their dimensions more than 0.1 mm, 0.2 mm, 0.3 mm, 0.4 mm, 0.5 mm, 0.6 mm, 0.7 mm, 0.8 mm, 0.9 mm or of more than 1 mm. Also excluded by a preferred embodiment are suspensions of tissue fragments which are not sprayable or which cannot be injected through a needle with an inner diameter of up to 0.8 mm without affecting the integrity of the tissue fragments.

As used herein, the term “donor tissue” refers to a tissue which has been obtained or which can be obtained from a living organism. The donor tissue does not include the organism and obtaining the donor tissue does not include sacrificing the organism. Typically, the donor tissue is either obtained from the patient (autograft) or from a donor (allograft) or from a biological sample comprising the donor tissue.

As used herein, “processing donor tissue”, “tissue procurement”, “polyhedron generation”, “polyhedron generating” or similar terms e.g., used with respect to preparing a tissue polyhedron suspension, means cutting donor tissue such as tissue explants, preferably by using rotating blades such as micro-surgery scalpel blades such as the blade assembly of the device of the present invention. Likewise, “procured tissue” or similar terms refer to the tissue processed from donor tissue using the device of the present invention.

As used herein, tissue fragments “obtainable by processing donor tissue” or “obtainable by processing tissue” and the like, define the tissue fragments by referring to a preferred process of the present invention by which it can be obtained. This, however, does not exclude that the tissue fragments have actually been obtained by another processes which does not include a step of cutting tissue. In other words, a tissue fragment which is “obtainable by processing tissue” of the skin, may actually be obtained by fragmenting the tissue by any other means such as exposing a biological sample comprising tissue to pressure or shearing forces.

In a preferred embodiment, the tissue fragments such as polyhedron-shaped tissue micro-transplants in the suspension of the present invention comprise at least 100 cells or comprise an average or mean of at least 100 cells.

In one embodiment, the tissue fragments such as polyhedron-shaped tissue micro-transplants of the tissue polyhedron suspension of the present invention have been obtained from a donor tissue. The term “donor tissue” as used herein includes tissue obtained directly from a subject or from tissue of a donor contained in a biological sample. Preferably, the suspension comprises substantially all cells or all cell types of said donor tissue. However, according to the present invention it is also envisaged to deplete certain cell types from the donor tissue in order to improve the treatment.

In one embodiment, the tissue polyhedron suspension of the present invention comprises single cells obtained from the donor tissue, wherein the ratio of cells in the tissue fragments such as polyhedron-shaped tissue micro-transplants and single cells is at least 10. The term “single cell” particularly refers to a cell that does not detectably associate with another cell. Preferred methods of determining the presence and/or amount of single cells include microscopy, H&E staining and microscopy, flow cytometry, and/or cell counting devices. According to the present teaching the term “at least 10” encompasses a ratio of for example 10 or more, 20 or more, 30 or more and even 100 or more. However, a ratio of “at least 10” also includes for example a ratio of up to 20, up to 30, up to 40, up to 50, up to 60, up to 70, or up to 80. This embodiment clarifies that the suspension of the present invention can comprise a mixture of single cells and tissue fragments and essentially limits the amount of single cells in the suspension to no more than 10% of the cells of the tissue fragments.

As used herein, the term “tissue polyhedron suspension” refers to tissue fragments such as polyhedron-shaped tissue micro-transplants which are suspended in an aqueous solution. Preferably, the aqueous solution is a physiologically acceptable solution comprising water, one or more salts and, optionally a buffer. Said solution is preferably selected from the group consisting of PBS, a physiological solution of sodium and potassium, a physiological solution of saline, a physiological solution of sodium, potassium magnesium and calcium, Ringer's solution, Ringer's lactate solution, physiological Hartmann solution, and a physiological solution comprising HEPES buffer.

The suspension of the present invention may comprise one or more additives. These additives may be added at any stage of production of the suspension of the present invention. Preferably, the additives are additives to increase stability of the tissue fragments in the suspension, to modify fluidity of the suspension, to increase the rate of proliferation of cells in the tissue fragments, or to improve healing of tissue in a patient. Additives include water, organic and inorganic salts, antibiotics, and/or regenerative factors including growth factors and cytokines.

In one embodiment, the tissue polyhedron suspension of the present invention is sprayable by a spray device and/or drippable by a dripping device. As used herein, a sprayable tissue polyhedron suspension is a suspension which can be transformed by a spray device into small airborne droplets, each comprising one or more of the tissue fragments such as polyhedron-shaped tissue micro-transplants of the suspension. Preferably, the spray device produces a droplet of a size sufficient to include or envelope the largest tissue fragment in the tissue polyhedron suspension of the present invention. Also preferred is that the spray device produces a minimal droplet size sufficient to include or envelope a tissue fragment of average size. The spray device can be any prior art spray device capable of producing the aforementioned droplets.

In one embodiment, the tissue polyhedron suspension of the present invention has not been prepared by a method comprising a step of chemical or enzymatic disruption or enzymatic digestion of tissue. The terms “chemical disruption” and “enzymatic disruption”, or “enzymatic digestion” refer to a process of tissue disruption by chemical or enzymatic means, it is essentially based on the destruction of interactions between neighboring cells and the end product of a chemical or enzymatic disruption of tissue is a single cell not interacting with a neighboring cell.

In one embodiment, the tissue polyhedron suspension does not contain an enzyme or chemical compound or has not been obtained by treating tissue with an enzyme or chemical compound. The term “enzyme” as used herein preferably refers to an enzyme, in particular to an enzyme that is added to a biological sample comprising tissue, wherein the enzyme is preferably selected from the group consisting of trypsin, dispase, preferably dispase I or dispase II, collagenase, thermolysin, pronase, hyaluronidase, elastase, papain, proteinase K, and pancreatin. The term “chemical compound” as used herein preferably refers to a chelating agent. The chelating agent is preferably selected from the group consisting of ethylenediaminetetraacetic acid (EDTA), ethylene glycol-bis(β-aminoethyl ether)-N,N,N′,N′-tetraacetic acid (EGTA), Fura-2, Fura-2AM, Indo-1 and 1,2-bis(o-aminophenoxy) ethane-N,N,N′,N′-tetraacetic acid (BAPTA).

The term “donor tissue” as used herein preferably refers to healthy tissue which may comprise cellular components and, optionally, non-cellular components. The donor tissue can be obtained from kind of tissue of a subject or patient. The donor tissue of the present invention preferably is or comprises tissue obtainable from roots of hair, mesenchymal tissue including dermis and fat, skin tissue, cartilage, bone, muscle, heart muscle, epithelial tissue, endothelial tissue, ectodermal tissue, mesodermal tissue, or endodermal tissue. Tissue and donor tissue can be obtained from a living organism or from a biological sample comprising the tissue or donor tissue. In a preferred embodiment, the tissue is an incomplete preparation of the donor tissue. An example of an incomplete preparation of skin is a tissue sample comprising the epidermis, but lacking the dermis. Another example, is an incomplete preparation of pancreas tissue comprising only the islet cells of the pancreas but not the vascular cells. Donor tissue may comprise cellular component and, optionally, non-cellular components such as bio-matrix components including proteins and proteoglycans, hormones, cytokines, mediators and/or growth factors.

In several aspects such as the second, third and fourth aspect, the present invention relates to a device for preparing the tissue polyhedron suspension of the present invention from a biological sample comprising donor tissue. The device of the present invention is a medical tissue processing device for generating polyhedron-shaped tissue micro-transplants, the device comprising a procurement chamber and a polyhedron generating tool.

The procurement chamber of the device of the present invention is a vessel. In one embodiment, the procurement chamber comprises a first end and a second end. In one embodiment, the first end comprises a first opening of the procurement chamber and, optionally, a means for closing the first opening. The second end is a closed end or comprises a second opening of the procurement chamber. In one embodiment, the second opening is at a height that allows removing tissue polyhedron suspension. In one embodiment, the second opening is in fluid communication with a means for reversibly closing the second opening, wherein said means for reversibly closing the second opening preferably comprises a Luer-lock cap, a Luer-lock stopcock, Luer-lock connectors or a tube clamping segment.

The procurement chamber may have a tubular shape. In one embodiment, the procurement chamber has a tubular shape and comprises a conical structure or a rounded structure at the second end. In one embodiment, the procurement chamber is made from a material that is resistant to the forces of centrifugation, preferably up to 20.000×g. As used herein, the term “up to 20.000×g” includes for example up to 15.000×g, up to 10.000×g, up to 9.000×g, or up to 8.000×g. In one embodiment, the procurement chamber is made from a material that is resistant to the mechanical forces of the polyhedron generating tool that is to be inserted into the lumen of the procurement chamber. In a preferred embodiment, the procurement chamber or the means for closing the first opening is made from a material that is selected from the group consisting of polycarbonate, polyvinylchloride, polypropylene, polyurethane, polyamide, polyethylene, polyethersulphone, polystyrole, polystyrene and silicone rubber.

In one embodiment, the procurement chamber of the present invention is or comprises a disposable sterile cell biology laboratory centrifugation tube, preferably of 50 to 250 ml.

In one embodiment, the procurement chamber of the present invention is or comprises a disposable sterile centrifugation tube that is modified with additional openings and/or configured to accept further means such as the tissue polyhedron suspension chamber and the liquid chamber described herein.

In one embodiment, the procurement chamber of the device of the present invention comprises a first end and a second end, wherein the first end comprises a first opening of the procurement chamber and the second end is a closed end and wherein optionally, the procurement chamber comprises a means for closing the first opening. This procurement chamber includes many of the commercially available centrifuge tubes currently used in hospitals or biological sciences. A typical example is a 15 ml or 50 ml Falcon™ tube.

In one embodiment, the procurement chamber of the device of the present invention comprises a first end and a second end, wherein the first end comprises a first opening of the procurement chamber. In one embodiment, the second end comprises a second opening of the procurement chamber. In one embodiment, the second opening is located near the second end of the procurement chamber. In one embodiment, the second opening is in fluid communication with a means for reversibly closing the second opening, wherein said means for reversibly closing the second opening preferably comprises a Luer-lock cap, a Luer-lock stopcock, Luer-lock connectors or a tube clamping segment. The fluid communication between the second opening and the means for reversibly closing the second opening may be established by a tubular structure which connects the second opening with the closing means. Alternatively, the closing means are directly connected to the second opening, i.e. without a connecting tubular structure. In one embodiment, the second opening is at a height that allows removing tissue polyhedron suspension from the procurement chamber.

In one embodiment, the procurement chamber of the device of the present invention comprises a third opening of the procurement chamber. In one embodiment, the third opening is located near the first end and/or near the first opening of the procurement chamber. In one embodiment, the third opening is located in the side wall of the procurement chamber. In one embodiment, the third opening is located near the first opening. The third opening may also be formed as an opening in the lid of the first opening, if present. For example, the lid of the first opening, if present, may comprise the third opening and/or a feed-through allowing to add material into the procurement chamber such as the fourth opening described herein. In one embodiment, the third opening is in fluid communication with a means for reversibly closing the third opening, wherein said means for reversibly closing the third opening preferably comprises a Luer-lock cap, a Luer-lock stopcock, Luer-lock connectors or a tube clamping segment. The fluid communication between the third opening and the means for reversibly closing the third opening may be established by a tubular structure which connects the third opening with the closing means. Alternatively, the closing means are directly connected to the third opening, i.e. without a connecting tubular structure. In one embodiment, the third opening is at a height that allows adding liquid into the procurement chamber.

The device of the present invention comprises a polyhedron generating tool which is a mixing and cutting tool. This tool is designed to be reversibly inserted into the lumen of the procurement chamber of the device of the present invention. The tool is typically inserted and removed through the first opening of the procurement chamber and, when placed in the lumen of the procurement chamber is capable to mix a suspension comprising tissue and/or tissue fragments and to cut the tissue and/or tissue fragments contained in the suspension. The polyhedron generating tool comprises at least one upper cutting blade and at least one lower cutting blade, such as micro-surgery scalpel blades, rotatably mounted on an agitating element. The agitating element may be formed as a shaft traversing at least a portion of the procurement chamber when the polyhedron generating tool is inserted therein.

Preferably, the lumen of the procurement chamber and/or the cutting and mixing tool are sterile. Preferably, the device is configured such that it allows maintaining sterile conditions within the procurement chamber while handling of the device, e.g, while operating the device such as described herein.

In one embodiment, the polyhedron generating tool comprises one or more additional cutting blades which are preferably arranged between the upper cutting blade and the lower cutting blade. For example, the polyhedron generating tool may comprise up to 2, up to 3, up to 4, up to 5, up to 6, up to 7, up to 8, up to 9, up to 10, up to 11, up to 12, up to 13, up to 14, up to 15, up to 16, up to 17, up to 18, up to 19, or up to 20 blades, preferably micro-surgery scalpel blades as described herein. For example, the polyhedron generating tool may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 blades, preferably micro-surgery scalpel blades as described herein. Preferably, the polyhedron generating tool comprises at least 4 blades. Thus, the polyhedron generating tool may comprise 2, 3, 4, 5, 6, 7, 8, 9, 10, 11, 12, 13, 14, 15, 16, 17, 18, 19, or 20 blades, preferably micro-surgery scalpel blades as described herein. The term “cutting blade” or simply “blade” includes blades of various materials and design. In a preferred embodiment, the cutting blade is a disposable blade. In one embodiment, the cutting blade is a reusable blade. In one embodiment, the cutting blade is a scalpel or comprises the blade of a scalpel. In one preferred embodiment, the cutting blade is made from a material selected from the group consisting of steel, stainless steel, tempered steel, high carbon steel, ceramic, titanium, diamond, sapphire, and obsidian. In one embodiment, the cutting blade assembly uses disposable surgical and/or micro-surgery scalpel blades. Preferably, the blades of the device of the present invention are disposable micro-surgery scalpel blades.

The blades are preferably rotatably mounted on an agitating element and spaced apart by at least one spacer element, the spacer element defining a space between the upper and lower cutting blade allowing for passage and cutting of the tissue in the procurement chamber, the agitating element further comprising an engagement portion that is adapted to be engaged by a drive mechanism, wherein the polyhedron generating tool, with its blade assembly, is configured to allow for the generation of fluidic turbulences and fluid movements of the suspension fluid during rotation of the upper and lower cutting blade, therefore the cutting side of the upper and lower cutting blade is positioned against the resultant direction of rotation of the biological sample in the fluid suspension.

The blade assembly in the device of the present application is such that tissue that is inserted into the device and processed using the blade assembly is swirled in the device due to the movement of the blade assembly and/or means for generating of fluidic turbulences and fluidic movements and is processed by the blades several times ultimately resulting in polyhedrons comprising a substantial number of living cells. During operating the device of the invention, movement of the blade assembly results in rotating layer-shaving of polyhedron-shaped tissue fragments tissue, where tissue passes vertically through the blade assembly and reenters the process multiple times, while changing of the horizontal orientation, with the result of precision-divide larger organ explants into micro polyhedrons for micro-transplantation. This is depicted in FIG. 7. The blade assembly preferably is a rotating sterile disposable micro-surgical scalpel assembly. Thus, the device of the present application represents a technology that avoids blunt tissue cutting, tissue grinding, chipping, mincing, or chopping by procuring tissue explants to polyhedron-shaped fragments for clinical transplantation. When used as described herein, the blade assembly of the device of the present invention results in obtaining tissue fragments that are in the form of polyhedrons. Operating the device of the present invention using donor tissue results in polyhedrons, wherein the natural cell population distribution of the donor tissue the polyhedrons are derived from is persevered. Likewise, the cell population embedding in original biomatrix arrangement is preserved. Therefore, also the levels of structure in tissue are preserved, e.g., on cellular level, biomatrix level, microvascular level and/or repetitive microvascular level. Importantly, the polyhedrons obtained by operating the device of the present invention comprise a substantial number of viable cells and preserve the natural adult stem cell niche found in the donor tissue.

“Viable cell” relates to an intact cell, i.e., a cell with an intact membrane that has not released its normal intracellular components such as enzymes, organelles, or genetic material. An intact cell preferably is a living cell capable of carrying out its normal metabolic functions.

“Substantial number of viable cells” as used herein means that at least 40% of the cells present in a polyhedron of the tissue polyhedron suspension of the present invention such as the tissue polyhedron suspension obtained by operating the device of the present invention are viable cells. In a preferred embodiment, the polyhedrons of the suspension of the tissue fragments comprise at least 40%, at least 45%, at least 50%, at least 55%, at least 60%, at least 65%, at least 70%, at least 75%, at least 80%, at least 85%, at least 90%, at least 95%, 96%, 97%, 98% or 99% viable cells.

In one embodiment, the means of the device for reversibly closing the second opening are in fluid communication with one or more layers of filtration membrane, which are preferably located between the second opening of the procurement chamber and the means for closing the second opening. In one embodiment, the means of the device for reversibly closing the third opening are in fluid communication with one or more layers of filtration membrane, which are preferably located between the third opening of the procurement chamber and the means for closing the third opening.

The membranes are preferably made from a material selected from the group consisting of polysulfone, polyethersulfone, polypropylene, polyamide, polytetrafluoroethylene (PTFE) and cellulose, preferably polyethersulfone. Preferably, the membranes have a mean pore diameter (MPD) that allows passage of the tissue fragments such as polyhedron-shaped tissue micro-transplants of the desired size but block passage of larger tissue fragments. In a preferred embodiment, the membranes have a mean pore diameter of 1000-4000 μm, preferably of at least 2000 μm and/or of up to 3000 μm.

In one embodiment, the procurement chamber of the present invention comprises at least one additional opening and a means for reversibly closing the additional opening, wherein the opening is preferably located in the side wall of the procurement chamber, preferably at a height that allows adding liquid or removing e.g. supernatant of centrifugation.

The means for reversibly closing the openings of the procurement chamber are preferably in fluid communication with one or more layers of filtration membrane. The membranes are preferably located between the opening of the procurement chamber and the means for reversibly closing the opening. The membranes are preferably made from a material selected from the group consisting of polysulfone, polyethersulfone, polypropylene, polyamide, polytetrafluoroethylene (PTFE) and cellulose, preferably polyethersulfone. Preferably, the membranes have a mean pore diameter (MPD) that allows passage of the fluid around the tissue fragments but block passage of the tissue fragments. In a preferred embodiment, the membranes have a mean pore diameter of 0.2-1 μm, preferably of at least 0.5 μm and of up to 100 μm. In another preferred embodiment, the membranes have a mean pore diameter of 10-100 μm.

The means for reversibly closing the second and/or third opening of the procurement chamber preferably comprise a Luer-lock cap, a Luer-lock stopcock, Luer-lock connectors or a tube clamping segment. The opening may allow for temporary insertion of pipettes or tubes introducing or removing liquid, preferably by automated actuation.

In one embodiment, the means of the present invention for closing the first opening is a closing lid for the procurement chamber that is connectable to the first end of the procurement chamber, and the polyhedron generating tool is arranged in the closing lid in such a way that it is rotatably mounted through a bore such as a central bore of the closing lid. Preferably, the agitating element of the polyhedron generating tool comprises an engagement portion configured to be engaged by a drive mechanism such as the motor of the drive unit of the invention or the hand operated drive mechanism of the drive unit of the invention.

In one embodiment, the closing lid for closing the first opening comprises a rotatably mounted first portion comprising a first feed-through and mounted on a second portion which when connected to the procurement chamber is in contact with the first opening and comprises a second feed-through of the same or different size, e.g., smaller or larger, preferably larger, than the first feed-through, wherein the rotatably mounted first portion allows to be rotated in at least a first position, wherein the first feed-through is superposed with the second feed-through thereby allowing to add material, e.g. donor tissue, into the procurement chamber, and at least a second position wherein the first feed-through is superposed with a continuous part of the second portion thereby allowing to close the lid such that no material can be added into the procurement chamber. The first and/or second feed-through is position between the centre and the edge of the lid and adjacent to the bore such as the central bore. A lid according to this embodiment is depicted in FIGS. 5c) and j).

Thus, in one embodiment, the procurement chamber of the device of the invention comprises a fourth opening which is optionally reversibly closable and preferably is formed in the lid of the first opening. The fourth opening may be provided with means for closing it. The fourth opening may allow adding material such as donor tissue into the procurement chamber, e.g., through the first opening. Using a lid for the first opening comprising a fourth opening formed in said lid allows inserting the polyhedron generating tool, e.g., through the first opening, and thereafter allows using the smaller fourth opening (i.e., partially closing the larger first opening by installing the lid comprising the fourth opening) for adding material such as donor tissue, followed by closing the fourth opening and thereby completely closing the first opening. Thus, said lid may further comprise the bore, as described herein. The lid of the first opening comprising a fourth opening is useful for providing and/or maintaining sterile medical conditions after assembly of the device, e.g., during operation of the device, because it allows adding material such as donor tissue through the smaller fourth opening (and therefore through the first opening but without the necessity to open it completely). For example, as described above, the fourth opening may be formed as a second feed-through in a second portion of the lid of the first opening, wherein the second portion of the lid, when connected to the procurement chamber, is in contact with the first opening, and may be closable by rotating a rotatably mounted first portion of the lid comprising a first feed-through, wherein the first portion is mounted on the second portion. In this embodiment, the fourth opening can be opened by rotating the first portion of the lid such that the first feed-through is superposed with the second feed-through, and can be closed by rotating the first portion of the lid such that the first feed-through is not superposed with the second feed-through, as described above. Alternatively, the fourth opening may be formed as a feed-through in a second portion of the lid of the first opening, wherein the second portion of the lid, when connected to the procurement chamber, is in contact with the first opening, as described above. In this alternative embodiment, the fourth opening may be closable by rotating a rotatably mounted first portion of the lid mounted on the second portion such that the first portion is superposed with the feed-through, thereby closing the fourth opening. In this embodiment, the first portion of the lid is configured such that it allows to be rotated in at least a first position, wherein the fourth opening is opened (e.g., the first portion is not superposed with the feed-through), and in at least a second position, wherein the fourth opening is closed (e.g., the first portion is superposed with the feed-through). In another embodiment, the fourth opening can be closed using a screw-on lid or a snap-on lid or another means of closing the fourth opening.

In one embodiment, the lid and the blades with the agitating element is a single unit or a single assembly that can be connected to a drive mechanism such as a motor, e.g., the drive mechanism of the drive unit of the invention.

In one embodiment, the lid, the blades, the blade assembly, the agitating element, the rod or shaft, the holding element, the liquid chamber, the tissue polyhedron suspension chamber and/or the procurement chamber are disposable.

In a preferred embodiment, the procurement chamber of the present invention has a tubular shape with an inner diameter of at least 1 cm or of up to 15 cm. At least 1 cm includes for example 1 to 15 cm or 2 to 6 cm. In another preferred embodiment, the procurement chamber of the present invention has a volume of 5 to 1000 ml, preferably of 50 to 250 ml. In one embodiment, the procurement chamber has a capacity for a fluid volume of up to 1000 ml. As used herein, the term “fluid volume of up to 1000 ml” includes for example a fluid volume of up to 750 ml, up to 500 ml, up to 400 ml, up to 300 ml, up to 200 ml, up to 100 ml, up to 50 ml, up to 10 ml, up to 5 ml, up to 1.5 ml or up to 1 ml. Preferably, the inner lumen of the procurement chamber comprises a void volume of up to 1000 cm³. As used herein, the term “void volume of up to 1000 cm³ includes for example a void volume of up to 750 cm³, up to 500 cm³, up to 400 cm³, up to 300 cm³, up to 200 cm³, up to 100 cm³, up to 50 cm³, up to 10 cm³, up to 5 cm³, up to 1.5 cm³ or up to 1 cm³.

In one embodiment, the procurement chamber of the present invention comprises a holding bracket that allows for automated transfer of the procurement chamber into and out of a centrifuge procurement chamber, supporting a semi-or fully automated device action, including computerized control.

In one embodiment, the device of the present invention comprises a tissue polyhedron suspension chamber configured to remove tissue polyhedron suspension from the procurement chamber. “Tissue polyhedron suspension chamber” refers to a vessel configured to accept a tissue polyhedron suspension such as the tissue polyhedron suspension of the invention. In one embodiment, the tissue polyhedron suspension chamber is removably connected to the procurement chamber of the device of the present application and can be removed from the device of the present application. The procurement chamber may comprise a portion for accepting the tissue polyhedron suspension chamber. In one embodiment, the tissue polyhedron suspension chamber is in fluid communication with the second opening of the procurement chamber of the device of the present application. Preferably, the tissue polyhedron suspension chamber is attached to the procurement chamber of the device of the present invention and optionally is disposable. Preferably, the tissue polyhedron suspension chamber is configured to allow removing tissue polyhedron suspension from the procurement chamber, e.g., during operation of the device, while maintaining sterile conditions within the tissue polyhedron suspension chamber and within the procurement chamber. Thus, in one embodiment, the tissue polyhedron suspension chamber is or comprises a sterile container such as a syringe such as a sterile medical syringe. The tissue polyhedron suspension chamber being a sterile container may allow operating the device comprising the tissue polyhedron suspension chamber without the need to sterilize the tissue polyhedron suspension obtained by said operating. Preferably, the tissue polyhedron suspension chamber is configured to be accepted by a tissue polyhedron suspension spray device. The presence of the tissue polyhedron suspension chamber allows removing the tissue polyhedron suspension from the procurement chamber during or following operation of the device of the invention in a fast and easy manner while maintaining sterile conditions such that the tissue polyhedron suspension received by the tissue polyhedron suspension chamber can immediately be used for medical application. This allows medical applications such as treating wounds to be faster and more efficient ultimately resulting in increased treatment efficacy and outcome as compared to techniques using devices known in the art.

In one embodiment, the device of the present invention comprises a liquid chamber configured to provide liquid into the procurement chamber. “Liquid chamber” refers to a vessel configured to accept a liquid. In one embodiment, the liquid chamber is removably connected to the procurement chamber of the device of the present application and can be removed from the device of the present application. The procurement chamber may comprise a portion for accepting the liquid chamber. In one embodiment, the liquid chamber is in fluid communication with the third opening of the procurement chamber of the device of the present application. Preferably, the liquid chamber is attached to the procurement chamber of the device of the present invention and optionally is disposable. Preferably, the liquid chamber is configured to allow adding liquid to the procurement chamber, e.g., during operation of the device, while maintaining sterile conditions within the liquid chamber and within the procurement chamber. Thus, in one embodiment, the liquid chamber is a sterile container such as a syringe such as a sterile medical syringe. In one embodiment, the liquid chamber is in fluid communication with the third opening, wherein one or more filtration membranes are position between the liquid chamber and the procurement chamber such that liquid added from the liquid chamber into the procurement chamber is filtered through the one or more filtration membranes. In one embodiment, while adding liquid from the liquid chamber into the procurement chamber, the filtration membranes allow sterile filtering the liquid. Thus, in one embodiment, the filtration membranes are membranes for sterile liquid filtration. Any membrane known in the art for sterile liquid filtration can be used. The liquid chamber being a sterile container (provided that the liquid filled into the liquid chamber is sterile liquid) and/or using one or more membranes for sterile liquid filtration as described may allow operating the device comprising the liquid chamber without the need to sterilize the tissue polyhedron suspension obtained by said operating and also eliminates problems arising from adding non-sterile liquid during preparation of the tissue polyhedron suspension. The presence of the liquid chamber allows adapting parameters such as viscosity, cell and fragment density, cell and fragment size, liquid osmolarity, liquid oncotic pressure, optochemical probes, temperature, pH of the suspension etc. of the tissue polyhedron suspension during operation of the device of the invention in a fast and easy manner without the need for further processing of the obtained suspension. Therefore, the quality and uniformity of the tissue polyhedron suspension can be drastically increased compared to techniques using art-known devices.

In one embodiment, the liquid chamber is used to add or supplement liquid during and/or subsequent to operating the device of the invention so as to dilute the tissue polyhedron suspension, manipulate its pH, osmolarity, viscosity, cell and fragment density, temperature etc. The liquid added during and/or subsequent to operating the device may be selected from the group consisting of buffers, preferably selected from the group consisting of phosphate buffered saline (PBS), a physiological solution of sodium and potassium, a physiological solution of saline, a physiological solution of sodium, potassium magnesium and calcium, Ringer's solution, Ringer's lactate solution, physiological Hartmann solution, and a physiological solution comprising HEPES buffer. Preferably, the liquid added using the liquid chamber is sterile and/or is added into the procurement chamber through at least one membrane for sterile liquid filtration as described herein. Thus, in one embodiment, the device of the invention comprises at least one membrane for sterile liquid filtration between the liquid chamber and the procurement chamber. Preferably, the liquid added using the liquid chamber is pharmaceutically and/or physiologically acceptable.

The present invention also relates to a drive unit, i.e., an assembly comprising a drive mechanism such as a motor, or a hand operated drive mechanism configured to engage the engagement portion of the agitating element of the polyhedron generating tool of the invention. In one embodiment, the drive unit is configured to accept the device of the invention. In one embodiment, the drive mechanism is configured to agitate the agitating element in horizontal and/or vertical direction. For example, the drive mechanism which is a motor is configured to provide acceleration, deceleration, velocity and/or vertical movement to the agitating element. For example, the hand operated drive mechanism is configured to provide acceleration, deceleration, velocity and/or vertical movement to the agitating element.

The motor of the drive unit of the invention may be an electrical motor. Any motor known in the art that is capable of moving, e.g., rotating, an agitating element and that is configurable to engage an engagement portion of an agitating element can be used.

The motor of the drive unit can be actuated by hand, e.g., by pressing a button starting the motor, or it can be controlled by computational means such as programmable means, e.g., based on signals received from the device with respect to rotational speed of the blades, amount of liquid within the procurement chamber, viscosity of the tissue polyhedron suspension, number of tissue fragments such as polyhedron-shaped tissue micro-transplants in the tissue polyhedron suspension, size of tissue fragments such as polyhedron-shaped tissue micro-transplants in the tissue polyhedron suspension etc. Using a drive unit actuated by computational means may improve pace of the method of preparing a tissue polyhedron suspension described herein and may also further improve the quality of the tissue polyhedron suspension obtained since parameters such as dilution of the suspension, viscosity of the suspension, size of the polyhedrons etc. can be controlled in a fast and precise manner.

In one embodiment, the drive mechanism which is or comprises a motor is configured to engage the engagement portion of the agitating element of the device of the invention comprising a tissue polyhedron suspension chamber, wherein the drive unit comprises automation means for automated removal of tissue polyhedron suspension from the procurement chamber of the device into the tissue polyhedron suspension chamber. In one embodiment, the hand operated drive mechanism is configured to engage the engagement portion of the agitating element of the device of the invention comprising a tissue polyhedron suspension chamber, wherein the drive unit comprises automation means for automated removal of tissue polyhedron suspension from the procurement chamber of the device into the tissue polyhedron suspension chamber. Automation means for automated removal of tissue polyhedron suspension comprise a motion means such as a motor, one or more pumps or a gear. In one embodiment, the motion means are configured to allow removing tissue polyhedron suspension from the procurement chamber into the tissue polyhedron suspension chamber, e.g., by providing negative pressure to the tissue polyhedron suspension chamber such that tissue polyhedron suspension is drawn into the tissue polyhedron suspension chamber. For example, the tissue polyhedron suspension chamber is a syringe such as a sterile medical syringe that may be disposable and the automation means are configured to move, e.g., elevate, the piston of the syringe during operation of the device such that liquid, e.g., tissue polyhedron suspension, is drawn from the procurement chamber of the device of the invention into the syringe. For example, the tissue polyhedron suspension chamber is a vessel and liquid, e.g., tissue polyhedron suspension, can be added using one or more pumps such as roller tube pumps, fingerprint tube pumps, piston pumps and/or plunger pumps and the automation means are configured to operate the one or more pumps during operation of the device such that liquid, e.g., tissue polyhedron suspension, is drawn from the procurement chamber of the device of the invention into the tissue polyhedron suspension chamber. Thus, in one embodiment, the automation means are configured to move a piston of a syringe. In one embodiment, the automation means are configured to operate one or more pumps.

In one embodiment, the device further comprises a liquid chamber and the drive unit preferably comprises automation means for automated addition of liquid from the liquid chamber into the procurement chamber. In one embodiment, the liquid chamber and/or the tissue polyhedron suspension chamber of the device are syringes as described herein and the automation means are configured to move the piston of the syringe(s). In one embodiment, the liquid chamber and/or the tissue polyhedron suspension chamber of the device are vessels and liquid can be added thereto and/or removed therefrom using one or more pumps such as roller tube pumps, fingerprint tube pumps, piston pumps and/or plunger pumps and the automation means are configured to operate the one or more pumps.

In one embodiment, the drive mechanism which is a motor is configured to engage the engagement portion of the agitating element of the device of the invention comprising a liquid chamber, wherein the drive unit comprises automation means for automated addition of liquid from the liquid chamber into the procurement chamber of the device. In one embodiment, the hand operated drive mechanism is configured to engage the engagement portion of the agitating element of the device of the invention comprising a liquid chamber, wherein the drive unit comprises automation means for automated addition of liquid from the liquid chamber into the procurement chamber of the device. Automation means for automated addition of liquid comprise a motion means such as a motor, one or more pumps or a gear. In one embodiment, the motion means are configured to allow adding liquid from the liquid chamber into the procurement chamber, e.g., by providing positive pressure to the liquid chamber such that liquid is pushed into the procurement chamber. While being added from the liquid chamber into the procurement chamber, the liquid optionally passes at least one membrane for sterile liquid filtration such that it is sterile filtered before entering the procurement chamber. For example, the liquid chamber configured to provide liquid into the procurement chamber of the device of the present invention is a syringe such as a sterile medical syringe that may be disposable and the automation means are configured to move, e.g., lower, the piston of the syringe during operation of the device such that liquid is provided from the syringe into the procurement chamber of the device of the invention. For example, the liquid chamber is a vessel and liquid can be removed therefrom using one or more pumps such as roller tube pumps, fingerprint tube pumps, piston pumps and/or plunger pumps and the automation means of the drive unit are configured to operate the pumps during operation of the device such that liquid is provided from the vessel into the procurement chamber of the device of the invention. Thus, in one embodiment, the automation means are configured to move a piston of a syringe. In one embodiment, the automation means are configured to operate one or more pumps.

In one embodiment, the device further comprises a tissue polyhedron suspension chamber and the drive unit preferably comprises automation means for automated removal of tissue polyhedron suspension from the procurement chamber into the tissue polyhedron suspension chamber. In one embodiment, the liquid chamber and/or the tissue polyhedron suspension chamber of the device are syringes as described herein and the automation means are configured to move the piston of the syringe(s). In one embodiment, the liquid chamber and/or the tissue polyhedron suspension chamber of the device are vessels and liquid can be added thereto and/or removed therefrom using one or more pumps such as roller tube pumps, fingerprint tube pumps, piston pumps and/or plunger pumps and the automation means are configured to operate the one or more pumps.

“Motion means” as used herein refers to any means capable of moving a fluid such as the liquid described herein or the tissue polyhedron suspension described herein into and/or out of a chamber such as the liquid chamber of the invention or the tissue polyhedron suspension chamber of the invention, e.g., by directly or indirectly exerting positive or negative pressure onto the fluid. Thus, in one embodiment, a motion means comprises a motor configured to move the piston of a syringe thereby exerting positive or negative pressure onto a fluid that thereby is drawn into or out of the syringe. A pump may exert positive or negative pressure onto a fluid that thereby is drawn into or out of a vessel or a chamber. Of course, the drive unit of the invention may also comprise motion means as described herein, wherein the motion means are not comprised by automation means but are actuated directly, e.g., by hand.

In one embodiment, the drive unit of the invention comprises operation control means configured to control acceleration, deceleration, velocity and/or vertical movement of the agitating element when the engaging portion is engaged by the drive mechanism which is a motor. In one embodiment, the drive unit of the invention comprises operation control means configured to control acceleration, deceleration, velocity and/or vertical movement of the agitating element when the engaging portion is engaged by the hand operated drive mechanism. “Operation control means” as used herein refers to any means, e.g., mechanical, pneumatic, electric or electronic, that allows controlling and altering parameters of a drive mechanism such as an electrical motor or a hand operated drive mechanism. In one embodiment, the operation control means are one or more electronic boards connected to an external control panel that is configured to be controlled from the outside of the drive unit and interfaced with the drive mechanism. In one embodiment, the operation control means are a gear and/or gear shifting. The operation control means preferably comprise means for direct or indirect user input to control and alter drive mechanism parameters, e.g., motor parameters or gear parameters, such as buttons, switches, selection wheels, gear shifting and so on. In one embodiment, the drive unit comprises a detection unit electronically connected to the drive mechanism and interfaced with the one or more electronic boards, wherein the detection unit is configured to detect at least one parameter of the tissue polyhedron suspension selected from viscosity, cell or fragment number, cell or fragment density, cell or fragment size, liquid osmolarity, liquid oncotic pressure, optochemical probes, temperature and pH. Thus, the drive unit may comprise probes allowing to monitor biochemical parameters of the suspension within the lumen of the procurement chamber.

As used herein, the terms “one or more” and “at least one” generally mean at least 1, at least 2, at least 3, at least 4, at least 5, at least 10, at least 15 or at least 50. In some cases, however, a limitation to up to 10, 20, 30, 40 or 50 may be desirable. For example, one or more pumps relates to at least 1 pump, at least 2 pumps, at least 3 pumps, at least 4 pumps, at least 5 pumps and so on.

In one aspect, the present invention relates to a method of preparing a medicinal suspension of polyhedron-shaped tissue micro-transplants (“tissue polyhedron suspension”). The method comprises a step of fragmenting a donor tissue, wherein the size or average size of the tissue fragment such as polyhedron-shaped tissue micro-transplants preferably comprises at least 100 cells. The donor tissue can be fragmented for example in any of the physiologically acceptable solutions described herein. Additionally, the physiologically acceptable solution may also be added after fragmenting the donor tissue. The terms “fragmenting donor tissue” or “fragmentation of donor tissue” means e.g. cutting donor tissue into tissue fragments, preferably into tissue fragments such as polyhedron-shaped tissue micro-transplants comprising at least 100 cells.

In one embodiment, the method for preparing the tissue polyhedron suspension of the present invention is based on the use of the device of the present invention. Accordingly, the present invention also discloses the use of the device of the invention for preparing the tissue polyhedron suspension described herein, which is preferably a sprayable tissue polyhedron suspension or a tissue polyhedron suspension which can be injected.

The method of preparing the tissue polyhedron suspension of the present invention preferably comprises a step (a) of adding a biological sample comprising donor tissue to the procurement chamber of the device of the present invention and, optionally, a step of adding a physiologically acceptable solution; and a step (b) of operating the device under conditions that result in a suspension comprising tissue fragments such as polyhedron-shaped tissue micro-transplants obtained from the donor tissue. Preferably, the tissue fragments of the suspension comprise at least 100 cells.

In one embodiment, the method of the present invention may comprise the additional step (c) of centrifuging the suspension resulting from step (b) to produce a sediment of tissue fragments such as polyhedron-shaped tissue micro-transplants and a supernatant; the additional step (d) of removing the supernatant from the sediment of tissue fragments of step (c); and the additional step (e) of suspending the sediment in a physiologically acceptable solution, thereby producing the suspension of the present invention comprising tissue fragments.

In one embodiment, the physiologically acceptable solution of the method of the present invention is selected from the group consisting of physiologically acceptable solution is selected from the group consisting (PBS), a physiological solution of sodium and potassium, a physiological solution of saline, a physiological solution of sodium, potassium magnesium and calcium, Ringer's solution, Ringer's lactate solution, physiological Hartmann solution, and a physiological solution comprising HEPES buffer. However, other physiologically acceptable solutions known to the skilled person may also be used. Further examples include tissue culture media such as RPMI, RPMI 1640, MEM, Opti-MEM, DMEM, DMEM/F12 and which may be serum free or supplemented with HSA, BSA, serum, 2-Mercaptoethanol, amino acids, vitamin solutions, glutamine, buffers, or sugar(s) including glucose, and the like. Furthermore, the media may be modified by adding or removing certain salts such as salts of calcium, magnesium, potassium, sodium.

In one embodiment, the method of the present invention may comprise the additional steps (c) of centrifuging the suspension resulting from step (b) in the presence of density-gradient centrifugation components such as Percoll® solution to produce a gradient sediment of tissue fragments such as polyhedron-shaped tissue micro-transplants a supernatants; (d) of removing the supernatant from the sediment of tissue fragments of step (c); and (e) of suspending a selected gradient sediment in a physiologically acceptable solution, thereby producing a suspension comprising tissue fragments of selected density.

In one embodiment, the method of preparing a tissue polyhedron suspension comprises a step of adding an agent that improves the quality of the suspension. Accordingly, the suspension of the present invention may comprise such agent. The agent can be a cellular component or non-cellular component. The agent may be an agent that increases the stability of the suspension by modifying the surface of the tissue fragments or an agent that modifies the fluidity of the suspension. The agent can be, for example, hydroxyl ethyl starch, water, glucose, cellulose, hyaluronic acid, collagen or a therapeutic agent such as an antibiotic or an anti-inflammatory agent such as a corticoid.

In one embodiment, the donor tissue used in the method of the present invention is or comprises roots of hair, mesenchymal tissue including dermis and fat, skin tissue, cartilage, bone, muscle, heart muscle, epithelial tissue, endothelial tissue, ectodermal tissue, mesodermal tissue, or endodermal tissue, and, optionally, bio-matrix components including proteins and proteoglycans, hormones, cytokines, mediators or growth factors. Thus, in one embodiment, the polyhedron-shaped tissue micro-transplants of the invention, e.g. as obtained by the method of the invention, comprise adult stem cells, stem cell supporting cells, organ-typical parenchymal cells, cell supporting biomatrix and cell supporting factors/mediators/cytokines/hormones.

The skin tissue may be obtained from any part of the body. A tissue sample comprising “skin tissue” may include cellular and non-cellular components of the epidermis, the dermis and/or the hypodermis. The term “skin” includes skin of the face, skin of the legs, arms, hands or feed, skin of the chest or of the back. The cellular components of the skin include for example Merkel cells, fibroblasts, keratinocytes, melanocytes, Langerhans cells, adipocytes, nerve cells, cells forming hair follicles, sweat glands, sebaceous glands, apocrine glands, lymphatic procurement chambers and blood procurement chambers.

The term “muscle tissue” as used herein refers to any kind of muscle tissue including cardiac muscle, smooth muscle and skeletal muscle. Muscle tissue can thus be obtained from a muscle biopsy, preferably a skeletal muscle biopsy. A tissue sample obtained from muscle tissue may comprises a cellular component and a non-cellular component. The cellular component includes cells such as myocytes and vascular endothelial cells. The non-cellular component includes for example collagens and hyaluronans.

The term “dermal tissue”, “dermis tissue” or “dermis” as used herein refers to the layer of skin between the epidermis and the hypodermis. A tissue sample of skin preferably comprises dermis tissue more preferably papillary dermis, reticular dermis and/or dermal papillae. In a preferred embodiment, a tissue sample comprising dermis may comprise cellular or non-cellular components of the dermis. The cellular components preferably include fibroblasts, macrophages, mast cells, cells forming receptors (e.g. nociceptors and thermoreceptors), cells forming hair follicles, sweat glands, sebaceous glands, apocrine glands, lymphatic procurement chambers and/or blood procurement chambers. The non-cellular components preferably include components of the extracellular matrix of the dermis such as collagen, lipids, collagen fibrils, microfibrils, elastic fibers, and/or hyaluronan. Also included are for example proteoglycans such as hyaluronan, versican and decorin and the like.

The term “cartilage” as used herein preferably refers to elastic cartilage, hyaline cartilage and fibrocartilage and includes cellular and non-cellular components. Accordingly, a tissues sample of cartilage may comprise cells such as chondrocytes, and non-cellular components such as collagen, proteoglycans (such as aggrecan) and for example hyaluronic acid.

In one embodiment, the tissue polyhedron suspension of the present invention is prepared by operating the device of the present invention under conditions that result in cutting the tissue into pieces or fragments of less than 3 mm, preferably less than 1 mm. The size of “less than 3 mm” and “less than 1 mm” preferably refers to the maximal size that is measurable at any dimension of a fragment. A tissue polyhedron suspension of less than 3 mm preferably does not, or not substantially, comprise fragments of larger sizes. Not substantially, as used here refers to an amount of up to 1%, calculated on the basis of total cell numbers. Preferably, the tissue fragment such as polyhedron-shaped tissue micro-transplants obtainable by the method comprises at least 100 cells. The term “less than 3 mm” preferably refers to a fragment of less than 2 mm, less than 1 mm, or less than 0.5 mm.

In one embodiment, the tissue polyhedron suspension which is prepared or obtainable by the method of the present invention is sprayable or can be injected, preferably through the skin into the target tissue using a surgical needle.

In a seventh aspect, the present invention relates to a tissue polyhedron suspension according to the present invention, which is obtained or obtainable from the method described herein.

In a eight aspect, the present invention relates to a method of treatment of a subject, preferably a patient in need of treatment. The subject can be any vertebrate or non-vertebrate, preferably the subject is a mammal, more preferably a human. The term subject includes a healthy subject and a subject in need of treatment, i.e. for example a subject affected from a pathological condition or an aesthetical issue desirable to be corrected, such as hair loss. The method can comprise one or more steps, in particular a step of administering the suspension to the subject or patient, wherein the delivery or administration is preferably by injection or by spraying. The suspension is either sprayed onto a particular tissue it is injected into a particular tissue, wherein the tissue is preferably a tissue in need of treatment.

The present invention also relates to the use of the tissue polyhedron suspension of the present invention for the preparation of a medicine or pharmaceutical composition for the treatment of a patient, preferably in a method comprising a step of administering the tissue polyhedron suspension, or a medicine or pharmaceutical composition comprising said tissue polyhedron suspension, to the patient. Furthermore, the present invention also relates to the resulting medicine or pharmaceutical composition as such. The medicine or pharmaceutical composition may comprise a documentation such as a summary of product characteristics (SPMC) and/or instructions for use. The medicine or pharmaceutical composition may also be packaged with other therapeutic agents or with means for administering the medicine or pharmaceutical composition to the subject or patient. Thus, the present invention also relates to a kit comprising the medicine or pharmaceutical composition and at least one additional agent, preferably a therapeutic agent and/or a means for administering the medicine or pharmaceutical composition to the subject or patient. Such means can be, for example, a spray device can be any device capable of delivering the sprayable suspension of the present invention to a tissue. Preferably, the device includes any of the spray devices described herein above.

In this aspect, the present invention also refers to the use of the tissue polyhedron suspension of the present invention for treating a subject or patient, preferably by a method comprising a step of administering the tissue polyhedron suspension, or a medicine or pharmaceutical composition comprising said tissue polyhedron suspension, to the subject or patient. The use can be any kind of use including for example a therapeutic use or a non-therapeutic use such as a cosmetic use. The use or treatment may thus comprise the treatment of a medical and non-classic medical-more wellness oriented-condition such as hair loss and wrinkles.

The method or use of the present invention may comprise a step of obtaining from a subject or from a biological sample of a subject donor tissue. The method or use may comprise a further step of preparing the tissue polyhedron suspension of the present invention from said donor tissue. In one embodiment, the suspension is prepared from donor tissue of the same subject also receiving the treatment, i.e. the donor is identical to the recipient. In one embodiment, however, the suspension is prepared from donor tissue of a subject not receiving the treatment. In this latter embodiment, the subject or patient is treated with a tissue polyhedron suspension prepared from donor tissue of another subject. The tissue or donor tissue can be any of the tissues or donor tissues described herein above. Preferably, the donor tissue has been obtained from a “donor site” in the subject, which corresponds to, or is identical with, the site of treatment or “recipient site”. The tissue of the donor site corresponds to, or is identical with, the tissue of the recipient site, provided a biological sample obtained from the donor site has substantially the same or at least similar cellular and non-cellular components as expected from a biological sample obtained from the recipient site in its healthy state. For example, donor tissue obtained from the upper side of the left arm would be considered to correspond to the healthy tissue of a recipient site located on the upper side of the right arm.

The tissue at the donor site preferably comprises or consists of healthy tissue. The term “healthy tissue” refers to tissue which is not affected from the condition for which the subject or patient is treated. In a preferred embodiment, the tissue at the donor site has no detectable predisposition to develop the condition from which the recipient site is affected. In one embodiment, the term “detectable predisposition” refers to a predisposition which is detectable in a tissue sample by determining one or more parameters such as a nucleotide sequence or a biochemical parameter such as a liver failure indicator, including albumin synthesis, or a clinical parameter such as alopecia.

In one embodiment, the patient is affected from a skin wound. The term “skin wound” as used herein refers to a wound of the skin such as a wound caused from a mechanical injury of the skin or a wound caused by an infection with a pathogen such as a virus, a bacterium or a fungus. Also comprised are skin wounds resulting from a surgical intervention. The term skin wound as used herein furthermore includes wounds caused by burns, incisions, lacerations, abrasions, avulsions, gunshot wounds, penetration wounds and hematomas. A skin wound may result from cancerous growth of cells or from a diabetic condition. The method of treatment of the present invention may thus require additional steps of treatment prior to the administration of the suspension of the present invention to the site of treatment. Such steps can typically include a surgical step and/or a step of cleaning of the wound, treatment with an antiseptic agent and/or treatment with an antibiotic agent. The skin wound may be an acute skin wound, a chronic skin wound, or an ulcer. The method preferably comprises spraying the tissue polyhedron suspension of the present invention onto the wound or ulcer. The term “burn” includes a burn caused for example by fire, a friction burn, a cold burn caused by skin freezing, a thermal burn, a radiation burn, a chemical burn or an electrical burn.

In one embodiment, the tissue polyhedron suspension of the present invention is administered by injection. Preferably the injection is an injection selected from the group consisting of intradermal injection, an intramuscular injection, subcutaneous injection, injection into the kidney, injection into the pancreas, and injection into the liver. Depending on the recipient tissue that is treated and the mode of injection that is required to reach the tissue, the suspension of the present invention is injected with an appropriate injection needle. Preferably, however, the injection needle has a minimal inner diameter of at least 0.4 mm to reduce the risk of damage of the tissue fragments such as polyhedron-shaped tissue micro-transplants in the suspension.

The invention also relates to a kit comprising the device of the invention and the drive unit of the invention. In one embodiment, the drive unit is configured to accept the device. In one embodiment, the kit comprises a device as described herein, and optionally a drive unit as described herein, wherein the kit further comprises one or more selected from the group of the following disposables, that are optionally sterile: a polyhedron generating tool, a group of blades, a blade assembly, a shaft, a tissue polyhedron suspension chamber, a liquid chamber, a tube for connecting a pump with a chamber as described herein. Moreover, a kit is envisaged comprising one or more selected from the group of the following disposables, that are optionally sterile: a polyhedron generating tool, a group of blades, a blade assembly, a shaft, a tissue polyhedron suspension chamber, a liquid chamber, a tube for connecting a pump with a chamber as described herein. Such a kit allows using the device of the invention, after a cleaning step, for the next patient without the need to sterilize the various parts of the device. This allows very fast re-use of the device of the invention, which is important, e.g., during field medicine or disaster medicine. In one embodiment, the kit further comprises a data carrier. The data carrier may be a non-electronical data carrier, e.g. a graphical data carrier such as an information leaflet, an information sheet, a bar code or an access code, or an electronical data carrier such as a floppy disk, a compact disk (CD), a digital versatile disk (DVD), a microchip or another semiconductor-based electronical data carrier. The access code may allow the access to a database, e.g. an internet database, a centralized, or a decentralized database. Said data carrier may comprise instructions for the use of the kit in the methods of the invention.

The present invention is further illustrated by the following examples which are not to be construed as limiting the scope of the invention.

### EXAMPLES

### Example 1

### Preparation of a Tissue Polyhedron Suspension from Muscle Tissue

A biological biopsy comprising tissue of choice is surgically removed, in this case a 1×1×1 cm muscle biopsy. A nurse standing next to the surgeon operating on the patient on the operation table is standing by, holding the sterile procurement chamber of the device of the invention in a way to ensure that all surfaces and materials associated with the content of the procurement chamber and all materials that could come into contact with the cells remain sterile. The sterile procurement chamber provides a volume of 50 ml when half filled, and it is wrapped in sterile single use disposable surgical drape, in a way the nurse can open the wrap temporarily from the outside of the drape.

The nurse is offering access to the lumen of the procurement chamber by temporary opening the wrap. The nurse pours 20 ml of sterile lactated Ringer's solution into the procurement chamber. The surgeon immerses the muscle sample into this Ringer's solution, using sterile forceps. The nurse takes the sterile lid of the procurement chamber that also includes the rotation blade cutting assembly, out of a second sterile wrap and uses it to close the procurement chamber. The nurse is closing the procurement chamber in a way the blade assembly is now within the procurement chamber. As one-step procurement, the nurse is operating the device to prepare the tissue polyhedron suspension. The nurse takes the procurement chamber with liquid and sample, pushes it into the motor assembly of the device in a way, the blade assembly connects to the motor, and lets the motor rotate the blade assembly in the liquid with the sample for 3 minutes. After that, the solution that now contains the micro cut tissue fragments of the original tissue biopsy (donor tissue) is emptied by the nurse into a 30 ml sterile Luer-lock syringe, and the syringe is handed over to the surgeon in a way maintaining sterility. Using a Luer-lock needle on the syringe, the surgeon now injects the solution with the tissue fragments (i.e. the micro-cut muscle tissue) into the target tissue.

This procurement is considered a minor manipulation that compares to a surgical preparation of a mesh-graft providing macro blades: no enzymes nor chemical tissue dissociation agents are involved. No storage of suspension prior to treatment and during treatment is involved. A transfer of the tissue polyhedron suspension into the procurement chamber of the device and then into the syringe is performed at room temperature, no cooling (e.g. on ice transfer) is involved.

### Example 2

### Patient in Need of Muscle Regeneration Treatment after Trauma with Muscle Loss, Application by Injection

A patient is in need of regenerative muscle therapy, after trauma with loss of muscle tissue. A surgeon removes a muscle biopsy surgically, in this case a 1×1×1 cm biopsy. The surgeon closes the open wound surgically with stiches, and uses wound dressings over the sutures. A nurse standing next to the surgeon operating on the patient on the operation table is standing by, holding the sterile procurement chamber of the device of the invention in a way ensuring that all surfaces and materials associated with the content of the procurement chamber and all materials that could come into contact with the cells remain sterile. The sterile procurement chamber provides a volume of 50 ml when half filled, and it is wrapped in sterile single use disposable surgical drape, in a way the nurse can open the wrap temporarily from the outside of the drape. The nurse is offering access to the lumen of the procurement chamber, by temporary opening the wrap. The nurse pours 20 ml of sterile lactated Ringer solution from a lactated Ringer's solution flask. The surgeon immerses the tissue sample into the Ringer's solution, using sterile forceps. The nurse takes the sterile lid of the procurement chamber that also includes the rotation blade cutting assembly, out of a second sterile wrap and uses it to close the procurement chamber. The nurse is closing the procurement chamber in a way the blade assembly is now within the procurement chamber. As one-step procurement, the nurse is operating the device to prepare a tissue polyhedron suspension. The nurse takes the procurement chamber with liquid and sample, pushes it into the motor assembly of the device in a way, the blade assembly connects to the motor, and lets the motor rotate the blade assembly in the liquid with the sample for 3 minutes. Subsequently, the solution that now contains the tissue fragments (i.e. micro cuts) of the original muscle tissue biopsy (donor tissue) is emptied by the nurse into a 30 ml sterile Luer-lock syringe, and the syringe is handed over to the surgeon in a way maintaining sterility. The surgeon disinfects the skin area over the target region under the skin, where the muscle loss is to be addressed. Using a Luer-lock needle on the syringe, the surgeon now injects the solution with the tissue fragments (i.e. the micro-cut muscle tissue) through the skin into the target tissue area, in one or several injections with various depth. The surgeon closes the wound by using a wound dressing over the injection area.

### Example 3

### Patient in Need of Hair Roots, Application by Injection

A patient is in need of regenerative hair therapy in front of his skull. A surgeon removes a skin biopsy surgically from a hairy skin area on an arm—as state of the art in surgery, in this case a 1×2 cm skin biopsy. The surgeon closes the open wound surgically with stiches, and uses wound dressings over the sutures. A nurse standing next to the surgeon operating on the patient on the operation table is standing by, holding the sterile procurement chamber of the device of the invention in a way ensuring that all surfaces and materials associated with the content of the procurement chamber and that could come into contact with the cells remain sterile. The sterile procurement chamber provides a volume of 50 ml when half-filled and it is wrapped in sterile single use disposable surgical drape, in a way the nurse can open the wrap temporarily from the outside of the drape. The nurse is offering access to the lumen of the procurement chamber, by temporary opening the wrap. The nurse pours 20 ml of sterile lactated Ringer's solution from a lactated Ringer's solution flask. The surgeon immerses the tissue into the Ringer's solution, using sterile forceps. The nurse takes the sterile lid of the procurement chamber that also includes the rotation blade cutting assembly, out of a second sterile wrap and uses it to close the procurement chamber. The nurse closes the procurement chamber in a way the blade assembly is now within the procurement chamber. As one-step procurement, the nurse is operating the device to prepare the tissue polyhedron suspension. The nurse takes the procurement chamber with liquid and sample, pushes it into the motor assembly of the device in a way, the blade assembly connects to the motor, and lets the motor rotate the blade assembly in the liquid with the sample for 3 minutes. After that, the solution that now contains the tissue fragments (i.e. micro cuts) of the original tissue biopsy (donor tissue) is emptied by the nurse into a 30 ml sterile Luer-lock syringe, and the syringe is handed over to the surgeon in a way maintaining sterility. The surgeon disinfects the skin area over the target region of the skin, where the hair loss is to be addressed. Using a Luer-lock needle on the syringe, the surgeon now injects the solution with the tissue fragments (i.e. micro-cut tissue) through under the skin into the target tissue area, in a depth where typically hair roots are located, he uses 0.2 ml liquid depositions in one injection and repeats this step as long as liquid remains in the syringe, with each injection site in proximity to the next, as expected from hair root distribution. The surgeon closes the wounds by using a wound dressing over the injection areas.

### Example 4

### Patient with Second Degree Thermal Burn

A patient is delivered with a serious skin wound resulting from a severe second degree thermal burn. Large areas of the left arm and of the left leg are affected. The surface area that is affected amounts to approximately 50 cm². The patient undergoes the routine burn wound treatment with debridement and wound observation over two days. Once the indication for a surgical grafting treatment is given, the patient is brought into a burn operation room and routine anaesthesia performed. The surgeon removes a skin biopsy of 1 cm×5 cm surgically as state of the art in surgery. The surgeon closes the open wound surgically with stiches, and uses wound dressings over the sutures. A nurse standing next to the surgeon operating on the patient on the operation table is standing by, holding the sterile procurement chamber of the device of the invention in a way, all surfaces and materials associated with the content of the procurement chamber and that could come into contact with the cells remain sterile. In the case of this example, the sterile procurement chamber provides a volume of 50 ml when half filled, and it is wrapped in sterile single use disposable surgical drape, in a way the nurse can open the wrap temporarily from the outside of the drape. The nurse is offering access to the lumen of the procurement chamber, by temporary opening the wrap. The nurse pours 20 ml of sterile lactated Ringer's solution from an OR lactated Ringer's solution flask. The surgeon immerses the tissue sample into the Ringer's solution, using sterile forceps. After that, the surgeon continues to prepare the burn wound by performing debridement, while the nurse uses the device of the invention. The nurse takes the sterile lid of the procurement chamber that also includes the rotation blade cutting assembly, out of a second sterile wrap and uses it to close the procurement chamber. The nurse is closing the procurement chamber in a way the blade assembly is now within the procurement chamber.

As one-step procurement, the nurse is operating the device to prepare the tissue polyhedron suspension. The nurse takes the procurement chamber with liquid and sample, pushes it into the motor assembly of the device in a way, the blade assembly connects to the motor, and lets the motor rotate the blade assembly in the liquid with the sample for 3 minutes. After that, the solution that now contains the tissue fragments (i.e. micro cuts) of the original skin tissue biopsy (donor tissue), is emptied by the nurse into a 30 ml sterile Luer-lock syringe, and the syringe is handed over to the surgeon in a way maintaining sterility. The surgeon sprays the solution over the open burn wounds in an even way by surface, distributing the solution evenly by volume on the left arm and the left leg, by using a cell spray suitable sprayer. After the suspension is sprayed on the wound, it presents in a way that no visible difference from prior to spraying is visible. The surgeon closes the wound by using a wound dressing over the spray grafting area as with mesh grafting, as first layer fatty gauze, as second layer gauze and as third layer a mechanically protective dressing. The wound is left under the dressing as with mesh-grafting. And inspections are performed as with mesh grafting. After visible re-epithelialization, no wound dressing is used and the wound treatment continues as with mesh grafting.

### Example 5

### Polyhedrons Obtained by the Method of the Present Invention Comprise Viable Cells

The idea of the micro-transplantation technology platform for tissue transplantation is to provide “3D Tissue Super-Structures” for regenerative medicine therapy. The procedure and device of the invention provide tissue micro-transplants for stimulation of regeneration in a wound and for tissue repair. They enable a therapy that bases on patients own cells (autologous) and includes regenerative progenitor cells. E.g. for regeneration of skin, joints, muscle, heart muscle, and reconstructive surgery. Using a patient's own tissue, the device of the invention harvests 3D tissue super-structures from the original tissue (autologous) for immediate micro-transplantation and self-regeneration. These harvested superstructures are polyhedron-shaped. They naturally preserve physiological vectors and host powerful information-cues that enable regeneration by the patients own cells after polyhedron transplantation. These vectors include the original tissues cells in their natural supportive environment, the connective tissue with growth factor and mediator information, and the “adult stem cell niches” with adult progenitor cells and their supporting cells enabling regeneration.

To demonstrate the ability of cells derived from the method herein, we performed examples using skin and generated polyhedron-shaped micro-transplants in in vitro models, to perform cell survival and cell behavior demonstrations in an in vitro model of skin wound healing where the cells were supposed to “repair an empty dish “wound” surface by migrating over it and restructuring a monolayer of epithelial skin cells, called re-epithelialization (FIGS. 9-17).

In a first in vitro study, pig skin derived polyhedrons were generated using the device of the invention to establish an in vitro model. This was followed by an in vitro study observing the behavior of resulting human skin cells in culture. In the first study, the micro-transplants were generated out of pig skin derived from fresh slaughterhouse samples. The polyhedrons were taken into in vitro culture Petri-dishes, to observe their culture behavior as an in vitro model of skin wound healing. The culture was performed in uncoated culture dishes using DMEM high glucose+10% FCS culture medium (DMEM) typically used for mesenchymal fibroblasts. Porcine keratinocyte culture was enabled best using DMEM medium, human keratinocyte medium with 10% FCS (KCM) did only support human keratinocytes. (FIG. 16: “3D tissue super-structures on day 14-16 of culture of human skin polyhedrons”).

From skin, at least two populations can be described: epidermal keratinocytes and mesenchymal fibroblasts, both were able to be transferred and the grew in microscopically (phase-contrast) visible clusters, as expected. In contrast to tissue fragments obtainable using prior art devices, the cells from the polyhedron-shaped micro transplants generated using the device of the invention reoriented themselves actively in the 3D space, after migrating out of the polyhedron, into a layer structure resembling the epithelial layer of the skin. They survived several weeks and could be passaged from dish to dish. Mesenchymal cells (that live under the epidermal layer) could be transferred as well. Cells were able to actively leave the polyhedron, migrate over the dish surface and then undergo mitoses to grow in number, towards forming a so-called monolayer on the dish surface. This was a first an in vitro proof of principle, of keratinocytes outgrowing from a polyhedron produced by the method of the invention using the device of the invention and repairing a wound by growing from the healthy edge of the wound towards the center of the wound and form there a layer of new keratinocytes, the so-called re-epithelialization. Devices of the prior art are not able to produce tissue polyhedron suspension harboring such capabilities.

In a second in vitro study, human skin derived tissue was used and polyhedrons generated in vitro. This was followed by an in vitro study using human skin cells to observe their culture behavior. The micro-transplants were generated with the described device in comparison to FIG. 9 in a smaller size and taken into in vitro culture Petri-dishes, to observe their culture behavior as an in vitro model of skin wound healing.

Skin samples (ca. 10 cm²) obtained funder informed consent from discarded material from human elective cosmetic eyelid surgery. They were immerged in 100 ml Ringer's Lactate solution in the device. Cutting blade spinning was performed at 20,000-30,000 rpm, for 20-40 sec. Polyhedrons were harvested and seeded in culture dishes for in vitro culture. Polyhedrons were seeded into uncoated or collagen-coated culture flasks using keratinocyte culture medium with fetal calf serum (FCS) or DMEM high glucose with FCS. N=3 repeats with individual human skin samples (ca. 10 cm² tissue area).

The study confirmed the results obtained with pig skin and showed that the device can obtain the anticipated size of the polyhedrons. (FIG. 10: “Culture of human skin polyhedrons-in vitro survival over two weeks and morphology”). Moreover, the active processes of adhesion, spreading, outgrowth, migration, division, re-epithelialization were demonstrated, indicating survival, viability, structural and functional integrity.

In a third study, primary cultures of the resulting polyhedrons were studied versus the outgrown cells out of primary polyhedron cultures which were then passaged into subsequent cultures. This was initiated to confirm comparable cell morphology in primary culture and follow-up culture. Also, this was performed in order to demonstrate that the resulting polyhedrons behave in in vitro culture comparably to conventional cultures of such cells. Cultures were performed in uncoated culture dishes using DMEM high glucose+10% FCS (DMEM) or keratinocyte culture medium+10% FCS (KCM) 10% FCS (KCM). (FIG. 11: “Culture of human skin polyhedrons”).

The results show that the cells actively leave the polyhedrons and are able to “repair” an empty dish surface by actively migrating over it, forming a mono-layer as desired for wound healing by re-epithelialization. The cells showed that they can perform such complex tasks and behaved as expected from conventional primary cultures. The polyhedrons where cultured in uncoated flasks with DMEM, high glucose+10% FCS. FIG. 12 “Human polyhedrons obtained according to the invention comprise viable cells” show human skin tissue polyhedrons at day 17 of culture. This and the results in FIGS. 13-17: show-as anticipated-that a monolayer of epithelial cells can comprise islets of cells of other cell-type morphology can outgrow from polyhedrons and that the cells are viable. Outgrowth and expansion of cells around polyhedrons towards active formation of large confluent keratinocyte mono-layers was regularly seen, including mitoses. This indicates that the skin derived cells from polyhedrons spontaneously start skin typical processes, re-epitelialization, and can conduct them.

1. A device for preparation of a suspension of polyhedron-shaped tissue micro-transplants (“tissue polyhedron suspension”) from a biological sample comprising donor tissue using rotating disposable precision micro-surgery scalpel blades, the device comprising:
a chamber (“procurement chamber”) for receiving and processing the donor tissue, the procurement chamber comprising
a first opening for providing tissue explants into the procurement chamber,
a second opening at a height that allows removing tissue polyhedron suspension from the procurement chamber, and
a third opening at a height that allows adding liquid into the procurement chamber; and
a polyhedron generating tool that is reversibly insertable into the procurement chamber through the first opening, the polyhedron generating tool comprising at least one upper micro-surgery disposable scalpel blade and at least one lower micro-surgery disposable scalpel blade rotatably mounted on an agitating element and spaced apart by at least one spacer element, the spacer element defining a space between the upper and lower micro-surgery scalpel blade allowing for passage and layer-shaving of the tissue in the procurement chamber into polyhedron-shaped tissue micro-transplants, the agitating element further comprising an engagement portion that is adapted to be engaged by a drive mechanism, wherein the polyhedron generating tool is configured to allow for the generation of fluidic turbulences and fluid movements of the suspension fluid during rotation of the upper and lower micro-surgery disposable scalpel blade, for a rotating layer-shaving, thereby the cutting side of the upper and lower micro-surgery scalpel blade is positioned against the resultant direction of rotation of the biological sample in the fluid suspension.

2. The device of claim 1, wherein the procurement chamber comprises a first and a second end, wherein the first end comprises the first opening.

3. The device of claim 2, wherein the second opening is located near the second end of the procurement chamber.

4. The device of claim 2 or 3, wherein the third opening is located near the first end of the procurement chamber.

5. The device of any one of claims 1 to 4, wherein the first opening comprises means for reversibly closing the first opening.

6. The device of claim 5, wherein the means for closing the first opening is a closing lid for the procurement chamber, wherein the polyhedron generating tool is arranged in the closing lid in such a way that it is rotatably mounted through a central bore of the closing lid.

7. The device of any one of claims 1 to 6, wherein the second opening is in fluid communication with a means for reversibly closing said opening.

8. The device of any one of claims 1 to 7, wherein the third opening is in fluid communication with a means for reversibly closing said opening.

9. The device of claim 7 or 8, wherein the means for reversibly closing the opening comprise a Luer-lock cap, a Luer-lock stopcock, Luer-lock connectors or a tube clamping segment.

10. The device of any one of claims 7 to 9, wherein the means for reversibly closing the opening are in fluid communication with one or more layers of filtration membrane, which are preferably located between the opening of the procurement chamber and the means for closing the opening.

11. The device of any one of claims 1 to 10, wherein the procurement chamber and/or the lid are disposable.

12. The device of any one of claims 1 to 11, wherein the procurement chamber has a tubular shape with an inner diameter of 1 to 15 cm, preferably of 2 to 6 cm.

13. The device of any one of claims 1 to 12, wherein the procurement chamber has a volume of 5 to 1000 ml, preferably of 50 to 250 ml.

14. The device of any one of claims 1 to 13, which comprises a holding bracket that allows for automated transfer of the procurement chamber into and out of a centrifuge.

15. The device of any one of claims 1 to 14, wherein the procurement chamber comprises at least one additional opening and a means for closing the additional opening, wherein the opening is located in the side wall of the procurement chamber, preferably at a height that allows removing supernatant of centrifugation and wherein the opening is optionally combined with a membrane.

16. The device of any one of claims 1 to 15, comprising a chamber (“tissue polyhedron suspension chamber”) configured to accept tissue polyhedron suspension from the procurement chamber.

17. The device of claim 16, wherein the tissue suspension chamber is removably connected to the procurement chamber.

18. The device of claim 17, wherein the tissue polyhedron suspension chamber is configured to remove tissue polyhedron suspension from the procurement chamber during operation of the device.

19. The device of any one of claims 16 to 18, wherein the tissue polyhedron suspension chamber is in fluid communication with the second opening of the procurement chamber.

20. The device of any one of claims 16 to 19, wherein the tissue polyhedron suspension chamber comprises a syringe or a vessel.

21. The device of any one of claims 1 to 20, comprising a chamber (“liquid chamber”) configured to provide liquid into the procurement chamber.

22. The device of claim 21, wherein the liquid chamber is removably connected to the procurement chamber.

23. The device of claim 21 or 22, wherein the liquid chamber is configured to provide liquid into the procurement chamber during operation of the device.

24. The device of any one of claims 21 to 23, wherein the liquid chamber is in fluid communication with the third opening of the procurement chamber.

25. The device of any one of claims 21 to 24, wherein the liquid provided by the liquid chamber into the procurement chamber comprises one or more buffer solutions.

26. The device of any one of claims 21 to 25, wherein the liquid chamber comprises a syringe or a vessel.

27. The device of any one of claims 20 to 26, wherein the syringe is a sterile medical syringe or the vessel is a sterile vessel, wherein the syringe or the vessel is preferably disposable.

28. The device of any one of claims 16 to 27, wherein the procurement chamber is configured to accept the tissue polyhedron suspension chamber.

29. The device of any one of claims 21 to 28, wherein the procurement chamber is configured to accept the liquid chamber.

30. The device of any one of claims 16 to 29, wherein the tissue polyhedron suspension chamber is configured to be accepted by a tissue suspension spray deposition, or drip deposition or injection deposition device.

31. The device of any one of claims 1 to 30, which is disposable and/or for one-way use.

32. The device of any one of claims 1 to 30, which is sterilizable and/or for multiple use.

33. The device of any one of claims 1 to 32, wherein the blades are directly mounted on the agitating element.

34. The device of any one of claims 1 to 33, wherein the blades are attached to a holding element which is mounted on the agitating element.

35. The device of any one of claims 1 to 34, wherein the agitating element comprises a rod or a shaft, which is optionally disposable.

36. The device of claim 34 or 35, wherein the holding element comprises a body with a cylindrical form or a cuboid form, wherein the blades protrude from the holding element.

37. The device of any one of claims 34 to 36, wherein the holding element comprises segments spacing apart the blades.

38. The device of any one of claims 1 to 37, wherein the polyhedron generating tool comprises 4, 6 or 8 blades.

39. The device of any one of claims 1 to 38, wherein the polyhedron generating tool comprises blades that are in beveled or tapered orientation with respect to the direction of rotation of the polyhedron generating tool.

40. The device of any one of claims 1 to 39, wherein the polyhedron generating tool comprises blades that, when the tool is seen in side view, are arranged such that their cutting edges are above their blunt edges and blades that arranged such that their cutting edges are below their blunt edges.

41. The device of any one of claims 1 to 40, wherein the polyhedron generating tool comprises blades that are arranged such that, when the tool is seen in side view, their cutting edges and blunt edges are horizontal.

42. The device of any one of claims 1 to 41, which comprises one or more pumps configured to add liquid into the procurement chamber of the device.

43. The device of any one of claims 1 to 42, which comprises one or more pumps configured to remove liquid from the procurement chamber of the device.

44. The device of any one of claims 16 to 43, which comprises one or more pumps configured to add liquid into the tissue polyhedron suspension chamber of the device.

45. The device of any one of claims 16 to 44, which comprises one or more pumps configured to remove liquid from the tissue polyhedron suspension chamber of the device.

46. The device of any one of claims 21 to 45, which comprises one or more pumps configured to add liquid into the liquid chamber of the device.

47. The device of any one of claims 21 to 46, which comprises one or more pumps configured to remove liquid from the liquid chamber of the device.

48. The device of any one of claims 42 to 47, wherein the one or more pumps are selected from the group consisting of roller tube pumps, fingerprint tube pumps, piston pumps and plunger pumps.

49. A drive unit comprising a drive mechanism, wherein the drive mechanism is configured to engage the engagement portion of the agitating element of the device of any one of claims 1 to 48.

50. The drive unit of claim 49, which is configured to accept the device of any one of claims 1 to 48.

51. A drive unit comprising a drive mechanism, wherein the drive mechanism is configured to engage the engagement portion of the agitating element of the device of any one of claims 16 to 48, wherein the drive unit is configured to accept the device, and
wherein the drive unit comprises automation means for automated removal of tissue suspension from the procurement chamber of the device into the tissue polyhedron suspension chamber.

52. A drive unit comprising a drive mechanism, wherein the drive mechanism is configured to engage the engagement portion of the agitating element of the device of any one of claims 21 to 48, wherein the drive unit is configured to accept the device, and
wherein the drive unit comprises automation means for automated addition of liquid from the liquid chamber into the procurement chamber of the device.

53. The drive unit of claim 51 or 52, wherein the automation means are configured to move a piston of a syringe.

54. The drive unit of claim 51 or 52, wherein the automation means are configured to operate one or more pumps selected from the group consisting of roller tube pumps, fingerprint tube pumps, piston pumps and/or plunger pumps.

55. The drive unit of any one of claims 49 to 54, wherein the drive mechanism is or comprises a motor, preferably an electrical motor.

56. The drive unit of any one of claims 49 to 54, wherein the drive mechanism is a hand operated drive mechanism, preferably is or comprises a gear.

57. The drive unit of any one of claims 49 to 56, comprising operation control means configured to control acceleration, deceleration, velocity and/or vertical movement of the agitating element when the engaging portion is engaged by the drive mechanism.

58. The drive unit of claim 57, wherein the operation control means are one or more electronic boards connected to an external control panel that is configured to be controlled from the outside of the drive unit and interfaced with the drive mechanism.

59. The drive unit of claim 58, comprising a detection unit electronically connected to the drive mechanism and interfaced with the one or more electronic boards, wherein the detection unit is configured to detect at least one parameter of the tissue polyhedron suspension selected from viscosity, cell or fragment number, cell or fragment density, cell or fragment size, liquid osmolarity, liquid oncotic pressure, optochemical probes, temperature and pH.

60. A method of preparing a medicinal suspension of polyhedron-shaped tissue micro-transplants (“tissue polyhedron suspension”), comprising a step of
(a) adding a biological sample comprising donor tissue to the procurement chamber of the device of any one of claims 1 to 48 and, optionally, adding a physiologically acceptable solution; and
(b) operating the device under conditions that result in a suspension comprising polyhedron-shaped tissue micro-transplants obtained from the donor tissue, wherein the tissue micro-transplants of the suspension preferably comprise an average number of cells of at least 100.

61. The method of claim 60, comprising the additional steps of
(c) centrifuging the suspension resulting from step (b) to produce a sediment of polyhedron-shaped tissue micro-transplants and a supernatant;
(d) removing the supernatant from the sediment of tissue micro-transplants of step (c); and
(e) suspending the sediment in a physiologically acceptable solution, thereby producing a suspension comprising tissue micro-transplants.

62. The method of claim 60 or 61, wherein the device comprises a liquid chamber configured to receive liquid and to provide the liquid into the procurement chamber, and wherein the method comprises adding liquid from the liquid chamber into the procurement chamber before or during step (b).

63. The method of any one of claims 62, wherein liquid is added to adapt a parameter of the tissue polyhedron suspension in the procurement chamber selected from the group consisting of viscosity, cell and fragment density, cell and fragment size, liquid osmolarity, liquid oncotic pressure, optochemical probes, temperature and pH of the suspension.

64. The method of claim 62 or 63, wherein the liquid added using the liquid chamber is a physiologically acceptable solution.

65. The method of any one of claims 60 to 64, wherein the device comprises a tissue suspension polyhedron chamber configured to accept tissue polyhedron suspension and to remove tissue polyhedron suspension from the procurement chamber into the tissue polyhedron suspension chamber, and wherein the method comprises removing tissue polyhedron suspension from the procurement chamber into the tissue polyhedron suspension chamber during or subsequent to step (b).

66. The method of any one of claims 60 to 65, wherein step (b) is performed using the drive unit of any one of claims 49 to 59.

67. The method of any one of claims 60 to 66, wherein the physiologically acceptable solution is selected from the group consisting of phosphate buffered saline, a physiological solution of sodium and potassium, a physiological solution of saline, a physiological solution of sodium, potassium magnesium and calcium, Ringer's solution, Ringer's lactate solution, physiological Hartmann solution, and a physiological solution comprising HEPES buffer.

68. The method of any one of claims 60 to 67, wherein the donor tissue is or comprises mesenchymal tissue including dermis and fat, cartilage, bone, muscle, skin tissue, epithelial tissue, endothelial tissue, ectodermal tissue, mesodermal tissue, endodermal tissue, or roots of hair and, optionally, bio-matrix components including proteins and proteoglycans, hormones, cytokines, mediators or growth factors.

69. The method of any one of claims 60 to 68, wherein the polyhedron-shaped tissue micro-transplants comprise viable cells of each cell type present in the donor tissue.

70. The method of any one of claims 60 to 69, wherein the polyhedron-shaped tissue micro-transplants adult stem cells, stem cell supporting cells, organ-typical parenchymal cells, cell supporting biomatrix and cell supporting factors/mediators/cytokines/hormones.

71. The method of claim 70, wherein the stem cells, supporting cells and biomatrix in the polyhedrons are organized in a way that reflects their organization in the donor tissue.

72. The method of any one of claims 60 to 71, wherein the device is operated to process the tissue into pieces of less than 3 mm, preferably less than 1 mm.

73. The method of any one of claims 60 to 72, wherein the tissue polyhedron suspension is sprayable and/or drippable.

74. A method of treatment of a patient comprising the step of administering a medicinal suspension of polyhedron-shaped tissue micro-transplants (“tissue polyhedron suspension”) obtained by the method of any one of claims 60 to 73 to the patient.

75. The method of claim 74, wherein the method comprises a step of obtaining from the patient a biological sample comprising donor tissue and a further step of preparing the tissue polyhedron suspension from said donor tissue of the patient.

76. The method of claim 75, wherein the method comprises a step of obtaining from a donor a biological sample comprising donor tissue and a further step of preparing the tissue polyhedron suspension from the donor tissue of the donor, wherein said donor is not the patient.

77. The method of any one of claims 74 to 76, wherein the tissue is or comprises mesenchymal tissue including dermis and fat, cartilage, bone, muscle, skin tissue, epithelial tissue, endothelial tissue, ectodermal tissue, mesodermal tissue, endodermal tissue, or roots of hair and, optionally, bio-matrix components including proteins and proteoglycans, hormones, cytokines, mediators or growth factors.

78. The method of any one of claims 74 to 77, wherein the tissue polyhedron suspension is administered by a spray-deposition device and wherein the tissue preferably is or comprises skin.

79. The method of claim 78, wherein the patient is affected from a skin wound, preferably an acute skin wound or a chronic skin wound, or an ulcer, and the method comprises spraying the tissue polyhedron suspension onto the wound or ulcer.

80. The method of claim 79, wherein the skin wound is a burn caused by fire, a friction burn, a cold burn caused by skin freezing, a thermal burn, a radiation burn, a chemical burn or an electrical burn.

81. The method of claim 79 or 80, wherein the wound receives an escharotomy prior to the spraying.

82. The method of claims 74 to 77, wherein the tissue polyhedron suspension is administered by injection and wherein the suspension is or comprises mesenchymal tissue including dermis and fat, cartilage, bone, muscle, epithelial tissue, endothelial tissue, ectodermal tissue, mesodermal tissue, endodermal tissue, or roots of hair and, optionally, bio-matrix components including proteins and proteoglycans, hormones, cytokines, mediators or growth factors.

83. The method of claim 76 or 77, comprising a further step of covering the treated wound with sheets such as oxygen permeable silicone synthetic rubber sheets, membranes such as polylactic membranes, gauze such as fatty cotton gauze, liquid removing bandages, negative pressure generating and/or elastic bandage.

84. A kit comprising the device of any one of claims 1 to 48 and the drive unit of any one of claims 49 to 59.

85. The kit of claim 84, further comprising instructions for using the device and the drive unit for performing the method of any one of claims 60 to 73.

86. A medicinal suspension of polyhedron-shaped tissue micro-transplants (“tissue polyhedron suspension”), wherein said tissue micro-transplants are obtainable by processing donor tissue and wherein the tissue micro-transplants in the suspension preferably comprise at least 100 cells.

87. A medicinal suspension of polyhedron-shaped tissue micro-transplants (“tissue polyhedron suspension”), wherein the tissue polyhedron suspension has been obtained by the method of any one of claims 60 to 73, preferably wherein the tissue micro-transplants in the suspension preferably comprise at least 100 cells.

88. The tissue polyhedron suspension of claim 86 or 87, wherein the tissue micro-transplants have been obtained from a donor tissue and wherein the suspension preferably comprises all cells or cell types of said donor tissue.

89. The tissue polyhedron suspension of claim 88, wherein the polyhedrons comprise viable cells of each cell type present in the donor tissue.

90. The tissue polyhedron suspension of claim 88 or 89, wherein the polyhedrons comprise stem cells, supporting cells and biomatrix.

91. The tissue polyhedron suspension of claim 90, wherein the stem cells, supporting cells and biomatrix in the polyhedrons are organized in a way that reflects their organization in the donor tissue.

92. The tissue polyhedron suspension of any one of claims 86 to 91, further comprising single cells obtained from the donor tissue, wherein the ratio of cells in the tissue micro-transplants and single cells is at least 10.

93. The tissue polyhedron suspension of any one of claims 86 to 92 which is a suspension in a physiologically acceptable solution, said solution preferably selected from the group consisting of PBS, a physiological solution of sodium and potassium, a physiological solution of saline, a physiological solution of sodium, potassium magnesium and calcium, Ringer's solution, Ringer's lactate solution, physiological Hartmann solution, and a physiological solution comprising HEPES buffer.

94. The tissue polyhedron suspension of any one of claims 86 to 93 which is sprayable by a spray device and/or drippable by a dripping device.

95. The tissue polyhedron suspension of any one of claims 86 to 94, wherein the suspension has not been prepared by chemical or enzymatic disruption of tissue and wherein the suspension preferably does not contain an enzyme or chemical compound, wherein the enzyme is selected from the group consisting of trypsin, dispase, preferably dispase I or dispase II, collagenase, thermolysin, pronase, hyaluronidase, elastase, papain, proteinase K, and pancreatin, and wherein the chemical compound is a chelating agent, preferably selected from the group consisting of ethylenediaminetetraacetic acid (EDTA), ethylene glycol-bis(β-aminoethyl ether)-N,N,N′,N′-tetraacetic acid (EGTA), Fura-2, Fura-2AM, Indo-1 and 1,2-bis(o-aminophenoxy) ethane-N,N,N′,N′-tetraacetic acid (BAPTA).

96. The tissue polyhedron suspension of any one of claims 86 to 95, wherein the donor tissue is or comprises mesenchymal tissue (including dermis and fat, cartilage, bone, muscle, skin tissue), epithelial tissue (including epidermal skin tissue), endothelial tissue (including vessels), ectodermal tissue, mesodermal tissue, endodermal tissue, or roots of hair and, optionally, bio-matrix components including proteins and proteoglycans, hormones, cytokines, mediators or growth factors.
